SEQUENCE LISTING (48 sequences)

SEQ ID NO: 1 - DNA, 18 bp, synthetic construct
  source 1..18: mol_type = other DNA
  misc_feature 1..18: note = MB1-P1 Mediator Primer
cgctctccgtgcccactc

SEQ ID NO: 2 - DNA, 18 bp, synthetic construct
  source 1..18: mol_type = other DNA
  misc_feature 1..18: note = MB1-P2 Mediator Primer
cagcgctctccgtgccca

SEQ ID NO: 3 - DNA, 18 bp, synthetic construct
  source 1..18: mol_type = other DNA
  misc_feature 1..18: note = MB1-P3 Mediator Primer
acactgtccagcgctctc

SEQ ID NO: 4 - DNA, 18 bp, synthetic construct
  source 1..18: mol_type = other DNA
  misc_feature 1..18: note = MB1-P4 Mediator Primer
ggtccacactgtccagcg

SEQ ID NO: 5 - DNA, 61 bp, synthetic construct
  source 1..61: mol_type = other DNA
  misc_feature 1..61: note = MB1 Molecular Beacon
  modified_base 1: mod_base = OTHER; note = Fluorescein Amidite (carboxyfluorescein)
  modified_base 61: mod_base = OTHER; note = Black Hole Quencher 1 (BHQ1)
cggcggagtgggcacggagagcgctggacagtgtggacccacgtctcgcagcaggccgccg

SEQ ID NO: 6 - DNA, 17 bp, synthetic construct
  source 1..17: mol_type = other DNA
  misc_feature 1..17: note = MB2-P1 Mediator Primer
acacttctttttgctcg

SEQ ID NO: 7 - DNA, 17 bp, synthetic construct
  source 1..17: mol_type = other DNA
  misc_feature 1..17: note = MB2-P2 Mediator Primer
tctcacacttctttttg

SEQ ID NO: 8 - DNA, 17 bp, synthetic construct
  source 1..17: mol_type = other DNA
  misc_feature 1..17: note = MB2-P3 Mediator Primer
cacctctcacacttctt

SEQ ID NO: 9 - DNA, 17 bp, synthetic construct
  source 1..17: mol_type = other DNA
  misc_feature 1..17: note = MB2-P4 Mediator Primer
atcacacctctcacact

SEQ ID NO: 10 - DNA, 36 bp, synthetic construct
  source 1..36: mol_type = other DNA
  misc_feature 1..36: note = MB2 Molecular Beacon
  modified_base 1: mod_base = OTHER; note = ROX (carboxy-X-rhodamine)
  modified_base 36: mod_base = OTHER; note = Black Hole Quencher 2 (BHQ2)
cgagcaaaaagaagtgtgagaggtgtgatgagctcg

SEQ ID NO: 11 - DNA, 20 bp, synthetic construct
  source 1..20: mol_type = other DNA
  misc_feature 1..20: note = HANDS universal tag sequence
gcaagccctcacgtagcgaa

SEQ ID NO: 12 - DNA, 36 bp, synthetic construct; identical to SEQ ID NO: 10
  source 1..36: mol_type = other DNA
  misc_feature 1..36: note = L-DJN001 Molecular Beacon
  modified_base 1: mod_base = OTHER; note = 5' 6-FAM (Fluorescein)
  modified_base 36: mod_base = OTHER; note = Black Hole Quencher 1 (BHQ1)
  modified_base 1..36: mod_base = OTHER; note = L-DNA

SEQ ID NO: 13 - DNA, 35 bp, synthetic construct
  source 1..35: mol_type = other DNA
  misc_feature 1..35: note = L-DJN002 Molecular Beacon
  modified_base 1: mod_base = OTHER; note = SUN fluorophore
  modified_base 35: mod_base = OTHER; note = Black Hole Quencher 2 (BHQ2)
  modified_base 1..35: mod_base = OTHER; note = L-DNA
gctgcaaaaaactcaacgatgtggaagtcagcagc

SEQ ID NO: 14 - DNA, 14 bp, synthetic construct
  source 1..14: mol_type = other DNA
  misc_feature 1..14: note = L-DJN003 Molecular Beacon
  modified_base 1..14: mod_base = OTHER; note = L-DNA
cgagctcatcacac

SEQ ID NO: 15 - DNA, 17 bp, synthetic construct
  source 1..17: mol_type = other DNA
  misc_feature 1..17: note = L-DJN004 Molecular Beacon
  modified_base 1..17: mod_base = OTHER; note = L-DNA
cgagctcatcacacctc

SEQ ID NO: 16 - DNA, 25 bp, synthetic construct
  source 1..25: mol_type = other DNA
  misc_feature 1..25: note = L-DJN005 Molecular Beacon
  modified_base 1..25: mod_base = OTHER; note = L-DNA
cgagctcatcacacctctcacactt

SEQ ID NO: 17 - DNA, 14 bp, synthetic construct
  source 1..14: mol_type = other DNA
  misc_feature 1..14: note = L-DJN006 Molecular Beacon
  modified_base 1..14: mod_base = OTHER; note = L-DNA
gctgctgacttcca

SEQ ID NO: 18 - DNA, 19 bp, synthetic construct
  source 1..19: mol_type = other DNA
  misc_feature 1..19: note = L-DJN007 Molecular Beacon
  modified_base 1..19: mod_base = OTHER; note = L-DNA
gctgctgacttccacatcg

SEQ ID NO: 19 - DNA, 25 bp, synthetic construct
  source 1..25: mol_type = other DNA
  misc_feature 1..25: note = L-DJN008 Molecular Beacon
  modified_base 1..25: mod_base = OTHER; note = L-DNA
gctgctgacttccacatcgttgagt

SEQ ID NO: 20 - DNA, 25 bp, synthetic construct
  source 1..25: mol_type = other DNA
  misc_feature 1..25: note = L-DJN001-A Quencher Beacon
  modified_base 1..25: mod_base = OTHER; note = L-DNA
  modified_base 25: mod_base = OTHER; note = Black Hole Quencher 1 (BHQ1)
aagtgtgagaggtgtgatgagctcg

SEQ ID NO: 21 - DNA, 25 bp, synthetic construct
  source 1..25: mol_type = other DNA
  misc_feature 1..25: note = L-DJN002-B Quencher Beacon
  modified_base 25: mod_base = OTHER; note = Black Hole Quencher 1 (BHQ1)
  modified_base 1..25: mod_base = OTHER; note = L-DNA
gctgctgacttccacatcgttgagt

SEQ ID NO: 22 - DNA, 14 bp, synthetic construct
  source 1..14: mol_type = other DNA
  misc_feature 1..14: note = L-DJN003-A1 Mediator Primer Beacon Pair
  modified_base 1..14: mod_base = OTHER; note = L-DNA
  modified_base 1: mod_base = OTHER; note = 5' 6-FAM (Fluorescein)
cgagctcatcacac

SEQ ID NO: 23 - DNA, 17 bp, synthetic construct
  source 1..17: mol_type = other DNA
  misc_feature 1..17: note = L-DJN004-A2 Mediator Primer Beacon Pair
  modified_base 1..17: mod_base = OTHER; note = L-DNA
  modified_base 1: mod_base = OTHER; note = 5' 6-FAM (Fluorescein)
cgagctcatcacacctc

SEQ ID NO: 24 - DNA, 25 bp, synthetic construct
  source 1..25: mol_type = other DNA
  misc_feature 1..25: note = L-DJN005-A3 Mediator Primer Beacon Pair
  modified_base 1..25: mod_base = OTHER; note = L-DNA
  modified_base 1: mod_base = OTHER; note = 5' 6-FAM (Fluorescein)
cgagctcatcacacctctcacactt

SEQ ID NO: 25 - DNA, 14 bp, synthetic construct
  source 1..14: mol_type = other DNA
  misc_feature 1..14: note = L-DJN006-B1 Mediator Primer Beacon Pair
  modified_base 1..14: mod_base = OTHER; note = L-DNA
  modified_base 1: mod_base = OTHER; note = SUN fluorophore
actcaacgatgtgg

SEQ ID NO: 26 - DNA, 19 bp, synthetic construct
  source 1..19: mol_type = other DNA
  misc_feature 1..19: note = L-DJN007-B2 Mediator Primer Beacon Pair
  modified_base 1: mod_base = OTHER; note = SUN fluorophore
  modified_base 1..19: mod_base = OTHER; note = L-DNA
actcaacgatgtggaagtc

SEQ ID NO: 27 - DNA, 25 bp, synthetic construct
  source 1..25: mol_type = other DNA
  misc_feature 1..25: note = L-DJN008-B3 Mediator Primer Beacon Pair
  modified_base 1..25: mod_base = OTHER; note = L-DNA
  modified_base 1: mod_base = OTHER; note = SUN fluorophore
actcaacgatgtggaagtcagcagc

SEQ ID NO: 28 - DNA, 49 bp, synthetic construct
  source 1..49: mol_type = other DNA
  misc_feature 1..49: note = L-DJN009 | Flu A | A-2 Mediator Probe
  modified_base 1: mod_base = OTHER; note = 5' 6-FAM (Fluorescein)
  modified_base 21^22: mod_base = OTHER; note = ZEN quencher
  modified_base 49: mod_base = OTHER; note = 3' Iowa Black FQ quencher
  modified_base 1..17: mod_base = OTHER; note = L-DNA
cgagctcatcacacctccttgtcacctctgactaagggaattttaggat

SEQ ID NO: 29 - DNA, 57 bp, synthetic construct
  source 1..57: mol_type = other DNA
  misc_feature 1..57: note = L-DJN010 | Flu A | A-3 Mediator Probe
  modified_base 1: mod_base = OTHER; note = 5' 6-FAM (Fluorescein)
  modified_base 57: mod_base = OTHER; note = 3' Iowa Black FQ quencher
  modified_base 29^30: mod_base = OTHER; note = ZEN quencher
  modified_base 1..25: mod_base = OTHER; note = L-DNA
cgagctcatcacacctctcacacttcttgtcacctctgactaagggaattttaggat

SEQ ID NO: 30 - DNA, 53 bp, synthetic construct
  source 1..53: mol_type = other DNA
  misc_feature 1..53: note = L-DJN011 | RSV A | A-2 Mediator Probe
  modified_base 1: mod_base = OTHER; note = 5' 6-FAM (Fluorescein)
  modified_base 53: mod_base = OTHER; note = 3' Iowa Black FQ quencher
  modified_base 21^22: mod_base = OTHER; note = ZEN quencher
  modified_base 1..17: mod_base = OTHER; note = L-DNA
cgagctcatcacacctcgctattgtgcactaargatatttgatgagaagtagt

SEQ ID NO: 31 - DNA, 55 bp, synthetic construct
  source 1..55: mol_type = other DNA
  misc_feature 1..55: note = L-DJN012 | RSV A | B-2 Mediator Probe
  modified_base 1: mod_base = OTHER; note = SUN fluorophore
  modified_base 1..19: mod_base = OTHER; note = L-DNA
  modified_base 23^24: mod_base = OTHER; note = ZEN quencher
  modified_base 55: mod_base = OTHER; note = 3' Iowa Black FQ quencher
actcaacgatgtggaagtcgctattgtgcactaargatatttgatgagaagtagt

SEQ ID NO: 32 - DNA, 41 bp, synthetic construct
  source 1..41: mol_type = other DNA
  misc_feature 1..41: note = L-DJN013 | SC2 | A-2 Mediator Probe
  modified_base 1: mod_base = OTHER; note = 5' 6-FAM (Fluorescein)
  modified_base 41: mod_base = OTHER; note = 3' Iowa Black FQ quencher
  modified_base 21^22: mod_base = OTHER; note = ZEN quencher
  modified_base 1..17: mod_base = OTHER; note = L-DNA
cgagctcatcacacctcaccccgcattacgtttggtggacc

SEQ ID NO: 33 - DNA, 38 bp, synthetic construct
  source 1..38: mol_type = other DNA
  misc_feature 1..38: note = L-DJN014 | SC2 | B-1 Mediator Probe
  modified_base 1..14: mod_base = OTHER; note = L-DNA
  modified_base 18^19: mod_base = OTHER; note = ZEN quencher
  modified_base 1: mod_base = OTHER; note = SUN fluorophore
  modified_base 38: mod_base = OTHER; note = 3' Iowa Black FQ quencher
actcaacgatgtggaccccgcattacgtttggtggacc

SEQ ID NO: 34 - DNA, 51 bp, synthetic construct
  source 1..51: mol_type = other DNA
  misc_feature 1..51: note = L-DJN015 | EV/RV | A-3 Mediator Probe
  modified_base 29^30: mod_base = OTHER; note = ZEN quencher
  modified_base 1: mod_base = OTHER; note = 5' 6-FAM (Fluorescein)
  modified_base 51: mod_base = OTHER; note = 3' Iowa Black FQ quencher
  modified_base 1..25: mod_base = OTHER; note = L-DNA
cgagctcatcacacctctcacacttcctccggcccctgaatgyggctaayc

SEQ ID NO: 35 - DNA, 45 bp, synthetic construct
  source 1..45: mol_type = other DNA
  misc_feature 1..45: note = L-DJN016 | EV/RV | B-2 Mediator Probe
  modified_base 1..19: mod_base = OTHER; note = L-DNA
  modified_base 45: mod_base = OTHER; note = 3' Iowa Black FQ quencher
  modified_base 1: mod_base = OTHER; note = SUN fluorophore
  modified_base 23^24: mod_base = OTHER; note = ZEN quencher
actcaacgatgtggaagtccctccggcccctgaatgyggctaayc

SEQ ID NO: 36 - DNA, 50 bp, synthetic construct
  source 1..50: mol_type = other DNA
  misc_feature 1..50: note = L-DJN017 | RSV B | B-1 Mediator Probe
  modified_base 1..14: mod_base = OTHER; note = L-DNA
  modified_base 50: mod_base = OTHER; note = 3' Iowa Black FQ quencher
  modified_base 1: mod_base = OTHER; note = SUN fluorophore
  modified_base 18^19: mod_base = OTHER; note = ZEN quencher
actcaacgatgtggtattccttactaaagatgtctgatkggaagtggtgg

SEQ ID NO: 37 - DNA, 55 bp, synthetic construct
  source 1..55: mol_type = other DNA
  misc_feature 1..55: note = L-DJN018 | RSV B | B-2 Mediator Probe
  modified_base 1..19: mod_base = OTHER; note = L-DNA
  modified_base 23^24: mod_base = OTHER; note = ZEN quencher
  modified_base 1: mod_base = OTHER; note = SUN fluorophore
  modified_base 55: mod_base = OTHER; note = 3' Iowa Black FQ quencher
actcaacgatgtggaagtctattccttactaaagatgtctgatkggaagtggtgg

SEQ ID NO: 38 - DNA, 39 bp, synthetic construct
  source 1..39: mol_type = other DNA
  misc_feature 1..39: note = L-DJN019 | Flu B | A-1 Mediator Probe
  modified_base 1..14: mod_base = OTHER; note = L-DNA
  modified_base 18^19: mod_base = OTHER; note = ZEN quencher
  modified_base 39: mod_base = OTHER; note = 3' Iowa Black FQ quencher
  modified_base 1: mod_base = OTHER; note = 5' 6-FAM (Fluorescein)
cgagctcatcacaccgagcagctgaaactgcggtgggag

SEQ ID NO: 39 - DNA, 44 bp, synthetic construct
  source 1..44: mol_type = other DNA
  misc_feature 1..44: note = L-DJN020 | Flu B | B-2 Mediator Probe
  modified_base 1..19: mod_base = OTHER; note = L-DNA
  modified_base 44: mod_base = OTHER; note = 3' Iowa Black FQ quencher
  modified_base 1: mod_base = OTHER; note = SUN fluorophore
  modified_base 23^24: mod_base = OTHER; note = ZEN quencher
actcaacgatgtggaagtccgagcagctgaaactgcggtgggag

SEQ ID NO: 40 - DNA, 57 bp, synthetic construct
  source 1..57: mol_type = other DNA
  misc_feature 1..57: note = L-DJN021 | HMPV | A-3 Mediator Probe
  modified_base 1..25: mod_base = OTHER; note = L-DNA
  modified_base 29^30: mod_base = OTHER; note = ZEN quencher
  modified_base 1: mod_base = OTHER; note = 5' 6-FAM (Fluorescein)
  modified_base 57: mod_base = OTHER; note = 3' Iowa Black FQ quencher
cgagctcatcacacctctcacacttaacygcagtgacaccytcatcattgcagcaag

SEQ ID NO: 41 - DNA, 46 bp, synthetic construct
  source 1..46: mol_type = other DNA
  misc_feature 1..46: note = L-DJN022 | HMPV | B-1 Mediator Probe
  modified_base 1..14: mod_base = OTHER; note = L-DNA
  modified_base 46: mod_base = OTHER; note = 3' Iowa Black FQ quencher
  modified_base 1: mod_base = OTHER; note = SUN fluorophore
  modified_base 18^19: mod_base = OTHER; note = ZEN quencher
actcaacgatgtggaacygcagtgacaccytcatcattgcagcaag

SEQ ID NO: 42 - DNA, 41 bp, synthetic construct
  source 1..41: mol_type = other DNA
  misc_feature 1..41: note = L-DJN023 | M. Pneumo | A-1 Mediator Probe
  modified_base 1..14: mod_base = OTHER; note = L-DNA
  modified_base 18^19: mod_base = OTHER; note = ZEN quencher
  modified_base 41: mod_base = OTHER; note = 3' Iowa Black FQ quencher
  modified_base 1: mod_base = OTHER; note = 5' 6-FAM (Fluorescein)
cgagctcatcacaccgctaacggcaacacgtaatcaggtca

SEQ ID NO: 43 - DNA, 44 bp, synthetic construct
  source 1..44: mol_type = other DNA
  misc_feature 1..44: note = L-DJN024 | M. Pneumo | A-2 Mediator Probe
  modified_base 1..17: mod_base = OTHER; note = L-DNA
  modified_base 44: mod_base = OTHER; note = 3' Iowa Black FQ quencher
  modified_base 1: mod_base = OTHER; note = 5' 6-FAM (Fluorescein)
  modified_base 21^22: mod_base = OTHER; note = ZEN quencher
cgagctcatcacacctccgctaacggcaacacgtaatcaggtca

SEQ ID NO: 44 - DNA, 43 bp, synthetic construct
  source 1..43: mol_type = other DNA
  misc_feature 1..43: note = L-DJN025 | C. Pneumo | A-1 Mediator Probe
  modified_base 1..14: mod_base = OTHER; note = L-DNA
  modified_base 43: mod_base = OTHER; note = 3' Iowa Black FQ quencher
  modified_base 18^19: mod_base = OTHER; note = ZEN quencher
  modified_base 1: mod_base = OTHER; note = 5' 6-FAM (Fluorescein)
cgagctcatcacacgaatggcaagtaggagcctctctatctta

SEQ ID NO: 45 - DNA, 44 bp, synthetic construct
  source 1..44: mol_type = other DNA
  misc_feature 1..44: note = L-DJN026 | C. Pneumo | B-1 Mediator Probe
  modified_base 1..15: mod_base = OTHER; note = L-DNA
  modified_base 1: mod_base = OTHER; note = SUN fluorophore
  modified_base 44: mod_base = OTHER; note = 3' Iowa Black FQ quencher
  modified_base 19^20: mod_base = OTHER; note = ZEN quencher
actcaacgatgtggcgaatggcaagtaggagcctctctatctta

SEQ ID NO: 46 - DNA, 38 bp, synthetic construct
  source 1..38: mol_type = other DNA
  misc_feature 1..38: note = L-DJN027 | B. Pert | A-1 Mediator Probe
  modified_base 1..14: mod_base = OTHER; note = L-DNA
  modified_base 18^19: mod_base = OTHER; note = ZEN quencher
  modified_base 1: mod_base = OTHER; note = 5' 6-FAM (Fluorescein)
  modified_base 38: mod_base = OTHER; note = 3' Iowa Black FQ quencher
cgagctcatcacaccaatccaacacggcatgaacgctc

SEQ ID NO: 47 - DNA, 49 bp, synthetic construct
  source 1..49: mol_type = other DNA
  misc_feature 1..49: note = L-DJN026 | B. Pert | A-3 Mediator Probe
  modified_base 1..25: mod_base = OTHER; note = L-DNA
  modified_base 29^30: mod_base = OTHER; note = ZEN quencher
  modified_base 49: mod_base = OTHER; note = 3' Iowa Black FQ quencher
  modified_base 1: mod_base = OTHER; note = 5' 6-FAM (Fluorescein)
cgagctcatcacacctctcacacttcaatccaacacggcatgaacgctc

SEQ ID NO: 48 - DNA, 47 bp, synthetic construct
  source 1..47: mol_type = other DNA
  misc_feature 1..47: note = L-DJN028 | RNase P | B-3 Mediator Probe
  modified_base 1..25: mod_base = OTHER; note = L-DNA
  modified_base 47: mod_base = OTHER; note = 3' Iowa Black FQ quencher
  modified_base 29^30: mod_base = OTHER; note = ZEN quencher
  modified_base 1: mod_base = OTHER; note = SUN fluorophore
actcaacgatgtggaagtcagcagctctgacctgaaggctctgcgcg

(19) United States
(12) Patent Application Publication
(10) Pub. No.: US 2026/0168016 A1
(43) Pub. Date: Jun. 18, 2026

(54) HIGH-ORDER TARGET DETECTION BY MULTI-DIMENSIONAL PCR

(71) Applicant: Vanderbilt University, Nashville, TN (US)

(72) Inventors: Frederick R. Haselton, Nashville, TN (US); Dalton Jay Nelson, Nashville, TN (US)

(21) Appl. No.: 19/366,976

(22) Filed: Oct. 23, 2025

Related U.S. Application Data
(60) Provisional application No. 63/710,672, filed on Oct. 23, 2024.

(51) Int. Cl. C12Q 1/686; C12Q 1/48; C12Q 1/6827; C12Q 1/6851; C12Q 1/689; C12Q 1/70

(52) U.S. Cl. CPC C12Q 1/686; C12Q 1/48; C12Q 1/6827; C12Q 1/6851; C12Q 1/689; C12Q 1/701; C12Y 207/07007; C12Q 2600/156; C12Q 2600/16

(57) ABSTRACT

Disclosed herein is the discovery of a combinatoric approach for multiplexed polymerase chain reaction assay methods for exponentially increasing the number of identifiable target nucleic acid sequences in a sample, wherein each target nucleic acid sequence has a unique signature element corresponding to a melting temperature for each fluorophore color channel.

### CROSS REFERENCE TO RELATED APPLICATIONS

This application claims the benefit of U.S. Provisional Patent Application 63/710,672, filed Oct. 23, 2024, the disclosure of which is hereby incorporated by reference in its entirety.

### GOVERNMENT SUPPORT CLAUSE

This invention was made with government support under Grant Number AI157827, awarded by the National Institutes of Health. The Government has certain rights in the invention.

### FIELD OF THE INVENTION

This invention is in the area of improvements in polymerase chain reaction-based methods for detecting one or more target nucleic acid sequences in a sample.

### INCORPORATION BY REFERENCE

The contents of the xml file named “10644-203US1_ST26.xml” which was created on Oct. 8, 2025, and is 126,119 bytes in size, are hereby incorporated by reference in their entirety.

### BACKGROUND OF THE INVENTION

Polymerase chain reaction (PCR) is one of the most widely used diagnostic technologies due to its unparalleled sensitivity and specificity. In infectious disease diagnostic applications, for example, PCR is the gold standard for determining the presence of a particular infectious pathogen. Multiplex PCR enables simultaneous detection of one or more pathogen target sequences in a single sample, broadening the diagnostic scope and conserving time and resources. Each additional PCR that can be incorporated into a multiplex reaction increases the utility of this gold standard approach. Assuming one could combine as many individual PCR reactions as desired into a single tube, state-of-the-art PCR instruments and detection reagents limit the maximum number of uniquely identifiable pathogens to the number of unique fluorescence channels of a given instrument. While commercially available multiplex PCR syndromic panels use complex approaches to overcome these inherent limitations (e.g., microfluidic separation of singleplex PCR reactions and special purpose instrumentation), these approaches are often too expensive to be routinely implemented in the clinic.

Novel PCR-based methods which enable highly multiplexed PCR reactions for testing many targets in a single reaction using standard real-time PCR instrumentation are urgently needed.

### SUMMARY OF THE INVENTION

It has been surprisingly and unexpectedly discovered that inclusion of multiple mediator probes for target nucleic acid sequences in a Polymerase chain reaction (PCR) assay allows for assigning a unique combinatoric code to each target nucleic acid sequence assayed, achieving high order PCR multiplexing using standard PCR instrumentation. The combinatoric approach described herein exponentially increases the number of identifiable targets in a sample of interest. An additional benefit is that the multi-bit PCR methods disclosed herein achieve high order multiplexing even with simpler PCR machines that have limited fluorescence channels or lower resolution melt analysis capabilities. The multiple mediator probe approaches described herein provide exponential scaling due to the combinatoric nature of the binary coding algorithm, surpassing the linear scaling limitations of previous implementations.

Multiplex PCR enables simultaneous detection of one or more target sequences, broadening the diagnostic scope and conserving time and resources. Multiplex PCR technologies, however, are often under-utilized because of certain technical limitations of conventional multiplex approaches and high costs associated therewith. Two notable challenges increase the difficulty of high order multiplexing

First, the limited fluorescent color channels for identifying targets by fluorescent means (e.g., hydrolysis probes) using high-quality standard or “conventional” instrumentation constrains the number of unique targets identifiable in a single multiplex reaction. PCR detection of a target and a corresponding signature event has a 1-to-1 relationship, which limits the number of discernable targets, for example to the number of unique fluorophore-melt temperature signature elements. By use of conventional multiplex PCR methods, the number of possible targets scales linearly with the number of identifiable sensing elements.

Second, as PCR multiplexing increases, the risk of interactions between the additional primer oligonucleotides (i.e., primer-dimers) increases, resulting in lower PCR efficiency and higher false positive rates. Users have implemented complex approaches such as specialized instrumentation and microfluidic separation of PCR reactions to combat some of these limitations. These approaches, however, are often cost-prohibitive.

In contrast, the methods disclosed herein, alternatively referred to as “multi-bit PCR” or “Multi-Dimensional PCR” herein, enable the unique identification of an exponential number of unique targets in a single sample. To enable the multi-bit PCR methods disclosed herein on standard PCR instrumentation, a novel encoding method is developed (a “combinatoric code”) to uniquely identify one PCR target among the many possible. The methods disclosed herein involve the creation of simultaneous sensing signature elements, or “bits”, for each target of interest which are used to create multi-bit combinatoric codes for each individual target nucleic acid sequence (see, e.g., FIG. 8A of Example 3). For example, when a PCR target is present, the collective fluorescence-Tm (F-Tm) signature element response sensed by the PCR instrument using the instrument's F-Tm sensing capabilities during a melt analysis corresponds to a unique identifier combinatoric code (see, e.g., FIG. 4 of Example 1). This allows for the capability of unambiguously identifying many targets in a complex mixture using standard PCR instrumentation (see, e.g., FIG. 8B of Example 3), but with fewer sensing elements.

As described herein, use of a reporter probe (e.g., a molecular beacon) and three unique mediator primers, enables generation of three clearly discernable and distinct signals with minimal variability (see, e.g., FIG. 5 of Example 2). Following this proof-of-concept study, use of the methods disclosed herein achieved consistent detection of three atypical bacteria (see, e.g., FIG. 6 of Example 2). The methods disclosed herein also achieved detection of and differentiation between pathogenic Alpha and Delta SARS-COV-2 variants (see, e.g., FIG. 7 of Example 2). Moreover, the introduction of additional reporter probe(s) led to the consistent detection of Influenza A targets (see, e.g., FIG. 8C of Example 3).

This improvement provides a significant advance in the state of the art of PCR-based multiplex methods.

In one aspect, provided herein is a method for detecting one or more target nucleic acid sequences in a sample by polymerase chain reaction (PCR) assay, comprising: (i) providing two or more mediator probes for each target nucleic acid sequence, wherein each of the two or more mediator probes comprise (a) a non-complementary mediator sequence, and (b) a target specific sequence which is complementary to a target binding site; (ii) contacting the sample, under conditions allowing for nucleic acid hybridization, with the mediator probes of step (i); (iii) contacting products of step (ii) with an enzyme having 5′ exonuclease activity to yield one or more 5′ flap sequences derived from the non-complementary mediator sequence; (iv) providing one or more detection probes and, under conditions allowing for nucleic acid hybridization, contacting the one or more 5′ flap sequences of step (iii) with the one or more detection probes; wherein each of the one or more detection probes comprises a capture sequence complementary to the 5′ flap sequence, or a fragment thereof; wherein each of the one or more detection probes are independently labeled with a reporter group capable of generating one or more signals in one or more fluorophore color channels; (v) contacting products of step (iv) with a nucleic acid polymerase to generate unique length double-stranded products; wherein each of the target nucleic acid sequences has a unique combinatoric code, wherein the unique combinatoric code comprises two or more signature elements each corresponding to a melting temperature (Tm) in a fluorophore color channel, and wherein the two or more signature elements are analyzable by the melting curve of the unique length double-stranded products of step (v) and the one or more signals of the reporter group of the detection probe; (vi) subjecting the unique length double-stranded products of step (v) to a melt and fluorescence analysis to determine a fluorescence-melting temperature (F-Tm) signature element in each of the one or more fluorophore color channels for each of the target nucleic acid sequences; and (vii) determining, according to the results of the melt and fluorescence analysis in step (vi), whether each of the one or more target nucleic acid sequences are present in the sample.

In one aspect, provided herein is a method for detecting one or more target nucleic acid sequences in a sample by polymerase chain reaction (PCR) assay, comprising: (i) providing two or more mediator probes for each target nucleic acid sequence, wherein each of the two or more mediator probes comprise (a) a non-complementary mediator sequence, and (b) a target specific sequence which is complementary to a target binding site; (ii) contacting the sample, under conditions allowing for nucleic acid hybridization, with the mediator probes of step (i); (iii) contacting products of step (ii) with an enzyme having 5′ exonuclease activity to yield one or more 5′ flap sequences derived from the non-complementary mediator sequence; (iv) providing one or more detection probes and, under conditions allowing for nucleic acid hybridization, contacting the one or more 5′ flap sequences of step (iii) with the one or more detection probes; wherein each of the one or more detection probes comprises (a) a capture sequence complementary to the 5′ flap sequence, or a fragment thereof, and (b) a templating sequence; wherein each of the one or more detection probes are independently labeled with a reporter group capable of generating one or more signals in one or more fluorophore color channels; (v) contacting products of step (iv) with a nucleic acid polymerase to generate unique length double-stranded products; wherein each of the target nucleic acid sequences has a unique combinatoric code, wherein the unique combinatoric code comprises two or more signature elements each corresponding to a melting temperature (Tm) in a fluorophore color channel, and wherein the two or more signature elements are analyzable by the melting curve of the unique length double-stranded products of step (v) and the one or more signals of the reporter group of the detection probe; (vi) subjecting the unique length double-stranded products of step (v) to a melt and fluorescence analysis to determine a fluorescence-melting temperature (F-Tm) signature element in each of the one or more fluorophore color channels for each of the target nucleic acid sequences; and (vii) determining, according to the results of the melt and fluorescence analysis in step (vi), whether each of the one or more target nucleic acid sequences are present in the sample.

In some embodiments, the method comprises detecting one or more target nucleic acid sequences among two or more potential assay targets in a sample.

In some embodiments, the one or more detection probes have a structure selected from a linear structure, a hairpin structure, a branching structure, a dendritic structure, a combination of hairpin and linear structure in a single strand, two strands in a molecular beacon double stranded DNA (dsDNA) structure, or a combination thereof.

In some embodiments, step (v) is performed under conditions allowing for an extension reaction. In some embodiments, step (v) is not performed under conditions allowing for an extension reaction. In some embodiments, step (v) is not performed under conditions allowing for an extension reaction, wherein the non-complementary mediator sequence alone, via a corresponding 5′ flap sequence, is capable of generating a signal.

In some embodiments, the one or more target nucleic acid sequences comprise DNA, RNA, or a mixture thereof.

In some embodiments, the one or more detection probes comprise reporter groups capable of generating a signal in 1, 2, 3, 4, or more than 4 fluorophore color channels. In some embodiments, the one or more detection probes comprise reporter groups capable of generating 1, 2, 3, 4, or more than 4 signals in each fluorophore color channel.

In some embodiments, several mediator probes are provided for a single target binding site of a target nucleic acid sequence. In some embodiments, several mediator probes are provided for several different target binding sites within the same target nucleic acid sequence.

In some embodiments, the reporter group comprises an intercalator dye. In some embodiments, the reporter group comprises a fluorophore.

In some embodiments, the method is performed in a single tube or well. In some embodiments, the method is performed in several tubes and/or wells.

In some embodiments, the non-complementary mediator sequence, the 5′ flap sequence, and/or the one or more detection probes comprise L-DNA or L-RNA residues.

In one aspect, provided herein is a method for detecting one or more target nucleic acid sequences in a sample by polymerase chain reaction (PCR) assay, comprising: (i) providing two or more mediator probes for each target nucleic acid sequence, wherein each of the two or more mediator probes comprise (a) a non-complementary mediator sequence independently labeled at the 5′ terminus with a reporter group, and (b) a target specific sequence which is complementary to a target binding site, (ii) contacting the sample, under conditions allowing for nucleic acid hybridization, with the mediator probes of step (i); (iii) contacting products of step (ii) with an enzyme having 5′ exonuclease activity to yield one or more 5′ flap sequences derived from the non-complementary mediator sequence; (iv) providing one or more quencher probes comprising a capture sequence complementary to the 5′ flap sequence, or a fragment thereof and, under conditions allowing for nucleic acid hybridization, contacting the one or more 5′ flap sequences of step (iii) with the one or more quencher probes; (v) contacting products of step (iv) with a nucleic acid polymerase to generate unique length double-stranded products; wherein each of the one or more target nucleic acid sequences has a unique combinatoric code, wherein the unique combinatoric code comprises two or more signature elements each corresponding to a melting temperature (Tm) in a fluorophore color channel, and wherein the two or more signature elements are analyzable by the melting curve of the unique length double-stranded products of step (v) and the one or more signals of the reporter group of the detection probe; (vi) subjecting the unique length double-stranded products of step (v) to a melt and fluorescence analysis to determine a fluorescence-melting temperature (F-Tm) signature element in each of the one or more fluorophore color channels for each of the one or more target nucleic acid sequences; and (vii) determining, according to the results of the melt and fluorescence analysis in step (vi), whether each of the one or more target nucleic acid sequences are present in the sample.

In some embodiments, the method comprises detecting one or more target nucleic acid sequences among two or more potential assay targets in a sample.

In one aspect, provided herein is a method for determining presence and/or amount of one or more target nucleic acid sequences derived from a virus or a bacterium in a sample, comprising: (i) designing one or more target specific sequences which are complementary to a target binding site in the one or more target nucleic acid sequences; and (ii) conducting the method of any aspect or embodiment disclosed herein using two or more mediator probes comprising the one or more target specific sequences of step (i).

In one aspect, provided herein is a method for determining presence of one or more single-nucleotide polymorphisms (SNPs) in one or more target nucleic acid sequences in a sample, comprising: (i) designing one or more target specific sequences to a target binding site comprising a potential SNP in the one or more target nucleic acid sequences; and (ii) conducting the method of any aspect or embodiment disclosed herein using two or more mediator probes comprising the one or more target specific sequences of step (i).

In one aspect, provided herein is a method for determining presence of one or more single-nucleotide polymorphisms (SNPs) in one or more target nucleic acid sequences in a sample, comprising: (i) designing one or more target specific sequences which are complementary to a target binding site comprising a potential SNP in the one or more target nucleic acid sequences; and (ii) conducting the method of any aspect or embodiment disclosed herein using two or more mediator probes comprising the one or more target specific sequences of step (i).

In one aspect, provided herein is a kit for use with a method of any aspect or embodiment described herein.

A summary of embodiments of the invention is described in further detail below.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 shows Multi-bit PCR encoding enables detection of exponentially more targets than single-bit approaches. The approach assigns multiple fluorophore channel-melt temperature elements to each target of interest. By encoding unique identifiers, multi-bit PCR increases the number of uniquely detectable targets. Here, 3 signals (left) combine for 7 unique target signals (left+right). In general, N elements can be combined into 2^{N}-1 codes. No template control (NTC) does not define a target.

FIG. 2 shows Taq polymerase nuclease activity is used to cleave 5′-flap sequences from mediator probes to initiate unique melt temperature (Tm) signatures. Different targets are detectable within a single fluorophore channel by designing target-specific mediator probes with concatenated mediator primer sequences. Once cleaved, these primers bind to a specific location on a molecular beacon reporter. Following polymerase extension, double-stranded products with unique length and characteristic Tm's are produced.

FIGS. 3A-3B show an overview of Homo-Tag Assisted Non-Dimer System (HANDS) as used in the multi-bit PCR methods disclosed herein.

FIG. 3A shows amplification of desired amplicon with universal homo-tag.

FIG. 3B shows prevention of primer-dimer accumulation through homo-tag induced hairpin formation.

FIG. 4 shows multi-bit PCR incorporates multiple mediator probes with unique mediator primer sequences (color tags) appended to detect a single target type. This results in characteristic fluorophore channel-melt temperature (Tm) signatures correspondent to the mediator primer sequences. With multiple mediator probes, fluorophore-Tm signatures (MP signals) can be used in combination to generate identifiers unique to a particular target. MP signals correspond to the fluorophore channel of universal molecular beacon reporters and the specific double-stranded sequence produced by the mediator primer. The unique identifiers are essentially a binary (on/off) code used to label an exponential number of targets within a single tube.

FIG. 5 shows the mediator primer and molecular beacon approach yields high performance as specific readout indicator. Mediator primers specific for three distinct regions on the molecular beacon reporter were extended by Taq DNA polymerase to generate unique length double-stranded products. The double stranded products resulted in consistent melt temperature that uniquely corresponds with each primer.

FIG. 6 shows the mediator probe PCR method consistently detects clinical management grouped atypical bacteria with a single molecular beacon reporter. Using a multiplex mediator probe PCR approach, three atypical bacteria, C. pneumoniae (dashed), M. pneumoniae (dotted), and B. pertussis (solid), each produce a unique melt temperature signature on the same molecular beacon. Signals are compared to the no target control (NTC; gray, solid). Each target was tested individually for three replicates (n=3 per target type).

FIG. 7 shows Ligation coupled with mediator probe facilitated PCR identifies characteristic SNVs for clinical SARS-COV-2 specimens. The SNV detection method consistently detects the E484 Alpha variant/control marker (top) and the L452R Delta variant characteristic marker (middle) in synthetic oligonucleotide targets by melt analysis as compared to no target controls (NTCs).

FIGS. 8A-8C show the potential for expanding identification of unique target nucleic acid sequences by melt temperature signature elements.

FIG. 8A shows design space (n) demonstrating an example of 15 melt temperature (Tm) and fluorescence combined labels. The existing approaches follow the standard paradigm of one output label for the detection of one target analyte (k=1). Multiple Tm-fluorescence labels are used in combination for detection of a single target, increasing the maximum detectable targets to n choose k, or 15 choose 2 or 3 in the pictured example.

FIG. 8B shows that the combinatoric approach exponentially increases the number of uniquely discernable targets within a given design space of the number, n, Tm and fluorescence signature elements.

FIG. 8C shows combinatoric approaches successfully detect Influenza A positive samples across titrated target concentrations. Combinatoric probe schemes varying the number of probes used (k=1, 2, or 3) were tested. Raw data is shown for each molecular beacon employed for the combinatoric signal output. The curves in the raw data correspond to the respective concentrations shown at the bottom of the figure.

FIG. 9 shows an exemplary L-/D-DNA chimeric mediator probe design as disclosed in Example 4 herein.

FIG. 10 shows an exemplary L-/D-DNA chimeric mediator probe design as disclosed in Example 4 herein.

FIG. 11 shows an exemplary L-/D-DNA chimeric mediator probe design as disclosed in Example 4 herein.

FIG. 12 shows an exemplary L-/D-DNA chimeric mediator probe design as disclosed in Example 4 herein.

FIG. 13 shows an exemplary L-/D-DNA chimeric mediator probe design as disclosed in Example 4 herein.

FIG. 14 shows the combinatoric PCR strategy is achieved by 5′-flap cleavage during the polymerase extension step of PCR. Mediator probes consisting of a target-specific region and a specific 5′ sequence called a mediator primer are used as a hydrolysis probe during PCR (1). Following cleavage, the mediator primers hybridize to and extend along molecular beacons (2). Extended products generate melt temperatures (Tm) with the beacon specific fluorescence. Tm-fluorescence signals are used in combination as target-unique signatures (3).

FIG. 15 shows multiple mediator probes corresponding to different signal outputs are used in combination, by either competition (left) or sequential (right) approaches, to exponentially increase the number of discernable targets within a single tube. Mediator probes (shown as probe 1 through probe 3) are designed to hybridize with the same target specific sequence while each containing unique 5′-flap sequences for generating Tm-fluorescence signals.

FIG. 16 shows mediator probes are designed by appending beacon-specific mediator primers to target-specific hydrolysis probes. Five mediator probes were designed for targeting Influenza A, each with unique mediator primers, to investigate the competition versus sequential combinatoric approaches.

FIG. 17 shows the five Influenza A specific mediator probes detect the virus specifically and with unique Tm-fluorescence outputs. For these studies, the mediator probes were used individually for detection of Influenza A and compared to no target controls.

FIG. 18 shows competition and sequential combinatoric approaches reliably detect Influenza A titrations. The combinatoric approaches each successfully detect Influenza A positive samples across titrated target concentrations. (Left) Combinatoric probe schemes varying the number of probes used (k=1, 2, or 3) and the competition (stacked) or sequential (side-by-side) approaches were tested. (Right) Raw data is shown for each of the three molecular beacons employed for the combinatoric signal output. The curves in the raw data correspond to the respective concentrations shown at the bottom of the figure.

FIG. 19 is a table showing a summary of the analytical performance of the probe design approaches with different number of probes (k). Influenza A virus (BEI NR-15241, (H1N1) pdm09, San Diego) was extracted and titrated. Following a separate reverse transcriptase step, the combinatoric approaches with k=2 and k=3 probes each were investigated for their analytical sensitivity. The shaded area is below the assay limit of detection. Viral load was determined by Hologic Panther RT-PCR with Influenza A quantitative standards.

FIGS. 20A-20C show the combinatoric approach enables future highly multiplex assay with minimal reporters and has practical utility even with reduced label design space.

FIG. 20A shows a simplified approach with two beacons, each with three robustly-spaced melt temperatures, for a proof-of-concept 6 choose 2 assay.

FIG. 20B shows the number of unique targets detectable measurably increases within the simplified assay as compared to the standard approach.

FIG. 20C shows a design grid demonstrating the 15-plex assay achievable through the 6 choose 2 combinatorics. This assay design is practical for panel-based assays, such as an in-development respiratory panel.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Definitions

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

As used in the specification and claims, the singular form “a”, “an”, and “the” include plural references unless the context clearly dictates otherwise. For example, the term “an agent” includes a plurality of agents, including mixtures thereof.

The terms “about” and “approximately” are defined as being “close to” as understood by one of ordinary skill in the art. In a non-limiting embodiment the terms are defined to be within 10%. In a non-limiting embodiment, the terms are defined to be within 5%. In a non-limiting embodiment, the terms are defined to be within 1%.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 10% (e.g., within 2% or 1%) of the particular value modified by the term “about.”

As used herein, the terms “optionally,” “may,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The terms “deoxyribonucleic acid” and “DNA” as used herein refer to a polymer composed of deoxyribonucleotides.

The terms “ribonucleic acid” and “RNA” as used herein refer to a polymer composed of ribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPSTM technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988).

The term “polynucleotide” refers to a single or double stranded polymer composed of nucleotide monomers.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

The terms “target nucleic acid sequence,” “target nucleic acid,” and “target sequence” interchangeably refer to a nucleic acid sequence of interest to be identified in a sample.

The terms “target binding site” or “target site” interchangeably refer to a nucleic acid sequence within a target nucleic acid sequence of interest to be identified in a sample which is complementary to a target specific sequence.

The term “mediator probe” as used herein refers to a nucleic acid probe comprising a target specific sequence and a mediator sequence that is cleaved by 5′ polymerase nuclease activity to release a 5′ flap sequence. In some embodiments, the mediator sequence is non-complementary to a target binding site of a target nucleic acid sequence. Thereby, upon contacting the mediator probe with a target nucleic acid sequence derived from a sample under conditions allowing for nucleic acid hybridization, the target specific sequence with hybridize with the target binding sequence to form a double-stranded structure, while the mediator sequence remains unhybridized in a single-stranded state. In some embodiments, the mediator probe is unlabeled. In some embodiments, the mediator probe is labeled.

The term “mediator sequence” as used herein refers to a nucleic acid sequence within the mediator probe which does not have complementarity to the target binding site or any site otherwise within a target nucleic acid sequence and which is cleaved by the 5′ exonuclease activity of a DNA polymerase enzyme as described herein. In some embodiments, the mediator sequence is 5′ to the target specific sequence of the mediator probe. In some embodiments, each target nucleic acid sequence has a corresponding unique mediator probe having a unique mediator sequence.

The term “target specific sequence” as used herein refers to a sequence within the mediator probe that has complementarity to and has specificity for a target binding site within a target nucleic acid sequence, and which can hybridize and/or anneal to the target binding site under conditions allowing for hybridization or annealing to form a double-stranded structure. In some embodiments, the target specific sequence is 3′ to the mediator sequence of the mediator probe. In some embodiments, the target specific sequence only hybridizes and/or anneals with a specific target nucleic acid sequence but does not hybridize and/or anneal with another nucleic acid sequence.

The terms “5′ flap sequence,” “mediator tag,” “mediator primer,” and “mediator” are used interchangeably herein to refer to the cleaved sequence product that results by the 5′ exonuclease cleavage of a single-stranded mediator sequence, which is not hybridized to the target nucleic acid sequence, via a DNA polymerase enzyme as described herein. A free 5′ flap sequence can then diffuse, contact, and hybridize with a complementary capture sequence of a detection probe to ultimately generate a signal.

The terms “targeting sequence,” “target-specific sequence,” and “target specific sequence” are used interchangeably herein to refer to a sequence within the mediator probe that is complementary or is otherwise guided by some means to bind a target binding site within a target nucleic acid sequence.

The term “under conditions allowing for nucleic acid hybridization” as used herein refers to variables in a reaction, including but not limited to ionic strength of buffer(s), pH, reagent components, temperature, etc., having suitable properties as to enable two nucleic acid molecules having complementary sequences to hybridize with one another.

The term “under conditions allowing for an extension reaction” as used herein refers to variables in a reaction, including but not limited to ionic strength of buffer(s), pH, reagent components, temperature, etc., having suitable properties as to enable a certain nucleic acid polymerase enzyme to use a strand of a nucleic acid molecule as a template to extend a strand of another nucleic acid molecule (e.g., primer) to form a double-stranded structure. In some embodiments, the nucleic acid polymerase enzyme is a DNA polymerase having 5′ exonuclease activity.

The terms “detection probe,” “fluorescent probe,” or “universal reporter” are used interchangeably herein to refer to an oligonucleotide molecule, separate from the one or more target nucleic acid sequences described herein, which are independently labeled with a reporter group capable of generating one or more signals.

The terms “capture sequence” or “mediator hybridization site” as used herein refer to a sequence within a detection probe having complementarity to the 5′ flap sequence as disclosed herein.

The terms “template sequence” or “templating sequence” as used interchangeably herein refers to a sequence with a detection probe as disclosed herein which can serve as a template for extending the 5′ flap sequence. In some embodiments, the template sequence is not complementary to a mediator probe, including but not limited to the mediator sequence or target specific sequence.

As used herein, the term “hybridize,” “hybridization,” “anneal,” and “annealing” are used interchangeably herein refer to the binding of one nucleic acid molecule to another nucleic acid molecule by the formation of hydrogen bonds between complementary bases between the two molecules.

As used herein, the term “complementary” refers to bases of one nucleic acid molecule forming a hydrogen bond to the corresponding bases of another nucleic acid molecule. Normally, the base adenine (A) is complementary to thymidine (T) and uracil (U), while cytosine (C) is complementary to guanine (G).

As used herein, the term “non complementary” as used herein refers to two nucleic acid sequences, for example including but not limited to a mediator sequence and a target nucleic acid sequence, which, under conditions allowing for nucleic acid hybridization cannot hybridize and/or anneal with each other to form a double-stranded structure.

The term “fluorophore” as used herein refers to a chemical compound, which when excited by exposure to a particular stimulus such as a defined wavelength of light, emits light (fluoresces), for example at a different wavelength (such as a longer wavelength of light). A detailed description of alternative fluorophores are provided herein.

The term “primer” as used herein refers to a nucleic acid molecule, such as a DNA oligonucleotide, for example sequences of at least 15 nucleotides, which can be annealed to a complementary target nucleic acid molecule by nucleic acid hybridization to form a hybrid complex between the primer and the target nucleic acid strand. A primer can be extended along the target nucleic acid molecule by a polymerase enzyme such as a PCR technique. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction.

The term “probe” refers to an isolated nucleic acid capable of hybridizing to a complementary sequence of a target nucleic acid. In some embodiments, a detectable label or reporter molecule is attached to a probe to enable detection of a target nucleic acid.

The term “fluorophore color channel” as used herein refers to optical components in a PCR machine that detect fluorescent signals emitted by fluorophores that label DNA or RNA during an amplification process. Each individual fluorophore color channel is tuned to a specific wavelength or range thereof, allowing for the differentiation of multiple fluorophores in a reaction.

The terms “signature element,” “fluorescence-melting temperature (F-Tm) signature element,” or “bits” are used interchangeably herein refers to a melting temperature (Tm) for each fluorophore color channel analyzable by the melting curve of unique length double-stranded products formed by contacting, in the presence of a nucleic acid polymerase, 5′ flap sequences and detection probes as described herein. As described herein, signature elements, alternatively referred to as “bits”, are assigned for each target nucleic acid sequence of interest can be used to create a multi-bit combinatoric code unique to each target nucleic acid sequence.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide. The terms “ribonucleic acid” and “RNA” as used herein mean a polymer composed of ribonucleotides.

The term “variant” means a polypeptide derived from a parent polypeptide by one or more (several) alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X,’ the insertion may be at position ‘X+1’ (‘downstream’) or at position ‘X−1’ (‘upstream’).

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full length polynucleotide or full length polypeptide.

A “nucleic acid variation” refers to an unknown nucleic acid sequence of interest that comprises at least one alteration when compared to its non-modified, reference nucleic acid sequence. Such “alterations” include, but are not limited to, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. As used herein, the terms “reduce”, “decrease”, “ablate”, and “eliminate” can be used interchangeably.

### L-DNA

L-DNA is the enantiomer of the natural D-DNA. L-Deoxyribose, the sugar backbone of L-DNA, is mirror of natural D-deoxyribose and has not been found in nature, despite the fact that other L-sugars (e.g., L-arabinose, L-lyxose, L-galactose, L-sorbose, and L-xylulose) do exist in nature. L-DNA can hybridize with complementary L-DNA sequences via classical Watson-Crick base-pairing, forming an L-DNA duplex much like a D-DNA duplex, except that the L-DNA duplex is a left-handed, mirror structure of the right-handed alpha-helix formed by D-DNA. Importantly, however, L-DNA does not hybridize with the complementary sequence on D-DNA backbones or D-RNA at physiological or even extreme (e.g., −60° C. or less) temperatures.

L-DNA is biologically inert and resistant to degradation. There are no natural nucleases that target and degrade L-DNA (Urata et al., 1991). Second, L-DNA is easily and inexpensively manufactured in large quantities. L-DNAs are synthesized using the same phosphoramidite chemistry that is used to synthesize DNA oligonucleotides. Third, and perhaps most significantly, L-DNA does not interact with the D-DNA present in the same solution. These properties enable the use of L-DNA templates as additives to a PCR reaction mix to serve as a perfect wild-type comparator during melt analysis.

Given that L-DNA is the enantiomer of D-DNA, the chemical reactivity of L-DNA is identical to D-DNA if the reactant is achiral. The biological reactivity of L-DNA with the chiral proteins and enzymes, however, is completely distinguishable from that of D-DNA. For example, L-DNA is not susceptible to natural DNA-modifying enzymes (e.g., ligases, polymerases and nucleases that can easily modify or degrade natural D-DNA). Taking advantage of these properties, L-DNA has been used to build intracellular nano-sensors as well as aptamers that exhibit enhanced serum stability. L-DNA has also been employed to construct a self-assembled DNA tetrahedral nanostructure showing high cellular uptake. L-DNA has also been adapted to PCR (see Adams et al., 2016) and used as a biostable DNAzyme with achiral metal ions. Various aspects and uses of L-DNA is reviewed in Young et al., 2019. L-DNA also has been observed to interact with intercalating dyes just the same as D-DNA (Malofsky et al., Anal. Chem. 2024, 96, 29, 11897-11905 (2024)).

### Melt Analysis

The term “melt analysis” as described herein is a method of detecting sequence variation based on the characteristic melt temperature (i.e., Tm) of a specific sequence. This melt temperature is defined as the reaction temperature at which 50% of the double-stranded DNA is dissociated into single strands. Melt temperatures on a highly calibrated instrument are provided as a feature of the instrument.

Melt analysis can also detect sequence variation based on the characteristic elapsed melt time of a specific sequence. This elapsed melt time is defined as the amount of elapsed time from the start of melt heat cycling up to the time at which 50% of the double-stranded DNA is dissociated into single strands. Elapsed melt time uses the property of standard PCR instruments to have a constant heat ramp source during the melt analysis.

The term “melting curve analysis” is an assessment of the dissociation characteristics of double-stranded DNA during heating. As the temperature is raised to and above the melt temperature of DNA, the double strand dissociates which leads to a rise in the absorbance of light at 260 nm as well as in hyperchromicity. The temperature at which 50% of the DNA strands are denatured is known as the melting temperature (otherwise referred to as “Tm,” “melting point,” or “melting peak,” and not to be confused with “elapsed melt time” or “tm”). Measurement of melting temperature can help characterize a PCR product as every dsDNA sequence has a characteristic melting temperature.

For example, the melt temperature information gathered can be used to infer the presence and identity of one or more SNPs, as G-C base pairing have 3 hydrogen bonds while A-T base pairs have only 2. DNA with a higher G-C content, for example due to SNPs, have a higher melting temperature than DNA with a higher A-T content. Base interactions with neighboring bases also affect the melt temperature.

Melt temperature information indicates a molecule's mode of interaction with DNA. For example, intercalators which slot in between base pairs and interact through pi stacking have a stabilizing effect on DNA structure and can lead to a rise in its melting temperature. The term “intercalator,” as used herein, refers to molecules capable of emitting a signal between the planar bases of deoxyribonucleic acid (DNA). In some embodiments, the intercalator is selected from ethidium bromide, SYBR® Green, SYBR® Safe, SYBR® Gold, LC Green, or LC Green Plus.

Also, increasing salt concentration diffuses negative phosphate repulsions in the backbone of DNA and leads to an increase in melting temperature. Conversely, pH can have a negative effect on the stability of DNA which may lead to a lowering of its melting temperature.

The energy required to break the base-base hydrogen bonding between two strands of DNA is dependent on the length of the DNA strands, GC content, as well as strand complementarity and “nearest neighbors.” By heating a reaction-mixture that contains double-stranded DNA sequences and measuring dissociation against temperature, these attributes can be inferred. Originally, strand dissociation was observed using UV absorbance measurements, but techniques based on fluorescence measurements are now the most common approach.

The temperature-dependent dissociation between two DNA-strands can be measured using a DNA-intercalating fluorophore (e.g., SYBR® green, EvaGreen®, etc.) or fluorophore-labeled DNA probes. In the case of SYBR® green (which fluoresces 1000-fold more intensely while intercalated in the minor groove of two strands of DNA), the dissociation of the DNA during heating is measurable by the large reduction in fluorescence. Alternatively, juxtapositioned probes (one featuring a fluorophore and the other, a suitable quencher; often referred to as “molecular beacons”) can be used to determine the complementarity of the probe to the target sequence when the strands are separated. Hybridization probes (or FRET probes) were also demonstrated to provide very specific melting curves from the single-stranded (ss) probe-to-amplicon hybrid.

Other double-strand specific dyes have since been developed (e.g., SYBR® Green), and probe-based melting curve analysis has become nearly ubiquitous. The probe-based technique is sensitive enough to detect single-nucleotide polymorphisms (SNP) and can distinguish between homozygous wildtype, heterozygous and homozygous mutant alleles by virtue of the dissociation patterns produced. Without probes, amplicon melting (melting and analysis of the entire PCR product) was not generally successful at finding single base variants through melting profiles. With higher resolution instruments and advanced dyes, amplicon melting analysis of one-base variants is now possible with several commercially available instruments. Such instruments include but are not limited to: Applied Biosystems 7500 Fast System, the 7900HT Fast Real-Time PCR System, Idaho Technology's LightScanner (the first plate-based high resolution melting device), Qiagen's Rotor Gene instruments, Roche's LightCycler 480 instruments, and Applied Biosystems™ QuantStudio™ 5 real-time PCR thermal cycler (Thermo Fisher Scientific #A28137).

While most real-time PCR machines have the option of melting curve generation and visualization, the level of analysis and software support varies. In general, they do not have high resolution capabilities. High Resolution Melt (known as either Hi-Res Melting, or HRM) is the advancement of this general technology and has begun to offer higher sensitivity for SNP detection within an entire dye-intercalating amplicon. It is less expensive and simpler in design to develop probeless melting curve systems. However, for genotyping applications, where large volumes of samples must be processed, the cost of development may be less important than the total throughput and ease of interpretation, thus favoring sequence probe-based genotyping methods.

### Methods

In one aspect, provided herein is a method for detecting one or more target nucleic acid sequences in a sample by polymerase chain reaction (PCR) assay, comprising: (i) providing two or more mediator probes for each target nucleic acid sequence, wherein each of the two or more mediator probes comprise (a) a non-complementary mediator sequence, and (b) a target specific sequence which is complementary to a target binding site; (ii) contacting the sample, under conditions allowing for nucleic acid hybridization, with the mediator probes of step (i); (iii) contacting products of step (ii) with an enzyme having 5′ exonuclease activity to yield one or more 5′ flap sequences derived from the non-complementary mediator sequence; (iv) providing one or more detection probes and, under conditions allowing for nucleic acid hybridization, contacting the one or more 5′ flap sequences of step (iii) with the one or more detection probes; wherein each of the one or more detection probes comprises a capture sequence complementary to the 5′ flap sequence, or a fragment thereof; wherein each of the one or more detection probes are independently labeled with a reporter group capable of generating one or more signals in one or more fluorophore color channels; (v) contacting products of step (iv) with a nucleic acid polymerase to generate unique length double-stranded products; wherein each of the target nucleic acid sequences has a unique combinatoric code, wherein the unique combinatoric code comprises two or more signature elements each corresponding to a melting temperature (Tm) in a fluorophore color channel, and wherein the two or more signature elements are analyzable by the melting curve of the unique length double-stranded products of step (v) and the one or more signals of the reporter group of the detection probe; (vi) subjecting the unique length double-stranded products of step (v) to a melt and fluorescence analysis to determine a fluorescence-melting temperature (F-Tm) signature element in each of the one or more fluorophore color channels for each of the target nucleic acid sequences; and (vii) determining, according to the results of the melt and fluorescence analysis in step (vi), whether each of the target nucleic acid sequences are present in the sample.

In some embodiments, the method comprises detecting one or more target nucleic acid sequences among two or more potential assay targets in a sample.

In some embodiments, each of the two or more mediator probes comprise, in 5′ to 3′ direction, (a) a non-complementary mediator sequence, and (b) a target specific sequence which is complementary to a target binding site.

In some embodiments, the method further comprises the step of determining the amount of each of the target nucleic acid sequences corresponding to the amplitude of each melting peak.

In some embodiments, the melt and fluorescence analysis of step (vi) comprises gradual heating and/or cooling of the unique length double-stranded products of step (v), wherein the one or more signals from the reporter group in each of the one or more detection probes is real-time monitored to provide the melting curve of each of the unique length double-stranded products of step (v), wherein identification of the F-Tm signature element for a unique length double-stranded product is determined according to a certain melting peak in the one or more fluorophore color channels, and wherein identification of the F-Tm signature element for an individual target nucleic acid sequence determines the presence of the individual target nucleic acid sequence in the sample.

In some embodiments, the melt and fluorescence analysis is performed from about 45° C. to about 95° C. at a ramp rate of about 1° C. per a duration of between about 1 second and about 10 seconds, for example about 1 second, about 2 seconds, about 3 seconds, about 4 seconds, about 5 seconds, about 6 seconds, about 7 seconds, about 8 seconds, about 9 seconds, or about 10 seconds. In some embodiments, the melt and fluorescence analysis is performed from about 45° C. to about 95° C. at a ramp rate of about 1° C. per a duration of about 5 seconds.

In some embodiments, each of the one or more detection probes comprises (a) one or more capture sequences complementary to the 5′ flap sequence, or a fragment thereof, and (b) a templating sequence.

In one aspect, provided herein is a method for detecting one or more target nucleic acid sequences in a sample by polymerase chain reaction (PCR) assay, comprising: (i) providing two or more mediator probes for each target nucleic acid sequence, wherein each of the two or more mediator probes comprise (a) a non-complementary mediator sequence, and (b) a target specific sequence which is complementary to a target binding site; (ii) contacting the sample, under conditions allowing for nucleic acid hybridization, with the mediator probes of step (i); (iii) contacting products of step (ii) with an enzyme having 5′ exonuclease activity to yield one or more 5′ flap sequences derived from the non-complementary mediator sequence; (iv) providing one or more detection probes and, under conditions allowing for nucleic acid hybridization, contacting the one or more 5′ flap sequences of step (iii) with the one or more detection probes; wherein each of the one or more detection probes comprises (a) a capture sequence complementary to the 5′ flap sequence, or a fragment thereof, and (b) a templating sequence; wherein each of the one or more detection probes are independently labeled with a reporter group capable of generating one or more signals in one or more fluorophore color channels; (v) contacting products of step (iv) with a nucleic acid polymerase to generate unique length double-stranded products; wherein each of the target nucleic acid sequences has a unique combinatoric code, wherein the unique combinatoric code comprises two or more signature elements each corresponding to a melting temperature (Tm) in a fluorophore color channel, and wherein the two or more signature elements are analyzable by the melting curve of the unique length double-stranded products of step (v) and the one or more signals of the reporter group of the detection probe; (vi) subjecting the unique length double-stranded products of step (v) to a melt and fluorescence analysis to determine a fluorescence-melting temperature (F-Tm) signature element in each of the one or more fluorophore color channels for each of the target nucleic acid sequences; and (vii) determining, according to the results of the melt and fluorescence analysis in step (vi), whether each of the target nucleic acid sequences are present in the sample.

In some embodiments, the method comprises detecting one or more target nucleic acid sequences among two or more potential assay targets in a sample.

In some embodiments, each of the one or more detection probes comprises, in 3′ to 5′ direction, (a) one or more capture sequences complementary to the 5′ flap sequence, or a fragment thereof, and (b) a templating sequence.

In some embodiments, each of the one or more detection probes do not comprise a templating sequence.

In one aspect, provided herein is a method for detecting one or more target nucleic acid sequences in a sample by polymerase chain reaction (PCR) assay, comprising: (i) providing two or more mediator probes for each target nucleic acid sequence, wherein each of the two or more mediator probes comprise (a) a non-complementary mediator sequence independently labeled at the 5′ terminus with a reporter group, and (b) a target specific sequence which is complementary to a target binding site, (ii) contacting the sample, under conditions allowing for nucleic acid hybridization, with the mediator probes of step (i); (iii) contacting products of step (ii) with an enzyme having 5′ exonuclease activity to yield one or more 5′ flap sequences derived from the non-complementary mediator sequence; (iv) providing one or more quencher probes comprising a capture sequence complementary to the 5′ flap sequence, or a fragment thereof and, under conditions allowing for nucleic acid hybridization, contacting the one or more 5′ flap sequences of step (iii) with the one or more quencher probes; (v) contacting products of step (iv) with a nucleic acid polymerase to generate unique length double-stranded products; wherein each of the target nucleic acid sequences has a unique combinatoric code, wherein the unique combinatoric code comprises two or more signature elements each corresponding to a melting temperature (Tm) in a fluorophore color channel, and wherein the two or more signature elements are analyzable by the melting curve of the unique length double-stranded products of step (v) and the one or more signals of the reporter group of the detection probe; (vi) subjecting the unique length double-stranded products of step (v) to a melt and fluorescence analysis to determine a fluorescence-melting temperature (F-Tm) signature element in each of the one or more fluorophore color channels for each of the target nucleic acid sequences; and (vii) determining, according to the results of the melt and fluorescence analysis in step (vi), whether each of the one or more target nucleic acid sequences are present in the sample.

In some embodiments, the method comprises detecting one or more target nucleic acid sequences among two or more potential assay targets in a sample.

In some embodiments, the non-complementary mediator sequence comprises L-DNA residues. In some embodiments, the non-complementary mediator sequence consists of L-DNA residues.

In some embodiments, the complementary target specific sequence comprises D-DNA residues. In some embodiments, the complementary target specific sequence consists of D-DNA residues.

In some embodiments, the non-complementary mediator sequence and complementary target specific sequence are separated by a reporter group or a quencher group. In some embodiments, the non-complementary mediator sequence and complementary target specific sequence are separated by a quencher group.

In some embodiments, the cleaved 5′ flap sequence comprises a mediator beacon. In some embodiments, the quencher probe comprises a quencher beacon.

In some embodiments, the mediator sequence comprises a sequence of any one of SEQ ID NOs: 1-4, 6-9, 22-27, or a sequence at least about 70% identical thereto, for example at least about 75% identical thereto, at least about 80% identical thereto, at least about 85% identical thereto, at least about 90% identical thereto, at least about 95% identical thereto, at least about 96% identical thereto, at least about 97% identical thereto, at least about 98% identical thereto, at least about 99% identical thereto, or more than 99% identical thereto.

In some embodiments, the detection probe comprises a molecular beacon. In some embodiments, the molecular beacon comprises a sequence of any one of SEQ ID NOs: 5, 10, 12-19, or a sequence at least about 70% identical thereto, for example at least about 75% identical thereto, at least about 80% identical thereto, at least about 85% identical thereto, at least about 90% identical thereto, at least about 95% identical thereto, at least about 96% identical thereto, at least about 97% identical thereto, at least about 98% identical thereto, at least about 99% identical thereto, or more than 99% identical thereto.

In some embodiments, the mediator beacon comprises a sequence of any one of SEQ ID NOs: 22-27, or a sequence at least about 70% identical thereto, for example at least about 75% identical thereto, at least about 80% identical thereto, at least about 85% identical thereto, at least about 90% identical thereto, at least about 95% identical thereto, at least about 96% identical thereto, at least about 97% identical thereto, at least about 98% identical thereto, at least about 99% identical thereto, or more than 99% identical thereto.

In some embodiments, the mediator beacon comprises a sequence of SEQ ID NO: 20 or SEQ ID NO: 21, or a sequence at least about 70% identical thereto, for example at least about 75% identical thereto, at least about 80% identical thereto, at least about 85% identical thereto, at least about 90% identical thereto, at least about 95% identical thereto, at least about 96% identical thereto, at least about 97% identical thereto, at least about 98% identical thereto, at least about 99% identical thereto, or more than 99% identical thereto.

In some embodiments, the one or more detection probes have a structure selected from a linear structure, a hairpin structure, a branching structure, a dendritic structure, a combination of hairpin and linear structure in a single strand, two strands in a molecular beacon double stranded DNA (dsDNA) structure, or a combination thereof. In some embodiments, the one or more detection probes are linear or have a hairpin structure. In some embodiments, the one or more detection probes have a branching/dendritic structure. In some embodiments, the one or more detection probes have combinations of hairpin and linear structure in a single strand. In some embodiments, the one or more detection probes comprise two strand in a molecular beacon dsDNA structure.

In some embodiments, the mediator probes have a 3′-OH end, or the 3′-end is blocked. In some embodiments, the 3′-end blocking is achieved by addition of a chemical moiety at the 3′-OH of the 3′ nucleotide of the mediator probe. In some embodiments, the chemical moiety comprises biotin, alkyl, or a minor groove binder (MGB) moiety. In some embodiments, the 3′-end blocking is achieved by removing the 3′—OH group of the 3′ nucleotide of the mediator probe. In some embodiments, the 3′-end blocking is achieved by replacing the 3′ nucleotide of the mediator probe with a dideoxynucleotide.

In some embodiments, step (v) is performed under conditions allowing for an extension reaction. In some embodiments, step (v) is not performed under conditions allowing for an extension reaction. In some embodiments, step (v) is not performed under conditions allowing for an extension reaction, wherein the non-complementary mediator sequence alone, via a corresponding 5′ flap sequence, is capable of generating a signal.

In some embodiments, the one or more target nucleic acid sequences comprise DNA, RNA, or a mixture thereof.

In some embodiments, the one or more detection probes comprise reporter groups capable of generating a signal in 2, 3, 4, or more than 4 fluorophore color channels. In some embodiments, the one or more detection probes comprise reporter groups capable of generating a signal in 2 or more fluorophore color channels. In some embodiments, the one or more detection probes comprise reporter groups capable of generating a signal in 3 or more fluorophore color channels. In some embodiments, the one or more detection probes comprise reporter groups capable of generating a signal in 4 or more fluorophore color channels.

In some embodiments, the one or more detection probes comprise reporter groups capable of generating 2, 3, 4, or more than 4 signals in each fluorophore color channel. In some embodiments, the one or more detection probes comprise reporter groups capable of generating 2 or more signals in each fluorophore color channel. In some embodiments, the one or more detection probes comprise reporter groups capable of generating 3 or more signals in each fluorophore color channel. In some embodiments, the one or more detection probes comprise reporter groups capable of generating 4 or more signals in each fluorophore color channel. In some embodiments, the one or more detection probes comprise reporter groups capable of generating 5 or more signals in each fluorophore color channel.

In some embodiments, several mediator probes are provided for a single target binding site of a target nucleic acid sequence.

In some embodiments, several mediator probes are provided for several different target binding sites within the same target nucleic acid sequence.

In some embodiments, one detection probe corresponds to between 2 and 6 mediator probes, for example 2, 3, 4, 5, or 6 mediator probes per detection probe.

In some embodiments, each mediator probe targets to an individual target nucleic acid sequence. In some embodiments, each of the target specific sequences comprised in all the mediator probes are different from each other.

In some embodiments, the enzyme having 5′ exonuclease activity is a DNA polymerase derived from a bacterium selected from Thermus aquaticus (Taq), Thermus thermophiles (Tth), Thermus filiformis, Thermis flavus, Thermococcus literalis, Thermus antranildanii, Thermus caldophllus, Thermus chliarophilus, Thermus flavus, Thermus igniterrae, Thermus lacteus, Thermus oshimai, Thermus ruber, Thermus rubens, Thermus scotoductus, Thermus silvanus, Thermus thermophilus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosipho africanus, Pyrococcus woesei, Pyrococcus horikoshii, Pyrococcus abyssi, Pyrodictium occultum, Aquifex pyrophilus or Aquifex aeolieus. In some embodiments, the enzyme having 5′ exonuclease activity is a Taq polymerase.

In some embodiments, the reporter group comprises an intercalator dye.

In some embodiments, the reporter group comprises a fluorophore.

In some embodiments, each of the one or more detection probes are independently labeled with (a) a reporter group capable of generating a signal, and (b) a quencher group capable of absorbing or quenching the signal generated by the reporter group.

In some embodiments, the reporter group is selected from 5′ 6-FAM, acridine orange, ALEX-350, CAL Fluor® Gold 540, CAL Fluor Orange 560, CAL Fluor Red 590, CAL Fluor Red 610, CAL Fluor Red 635, CY3, CY5, CY5.5, ethidium bromide, EvaGreen, EvaRuby, FAM, HEX, JOE, MAX, PicoGreen, Quasar 670, Quasar 705, ROX, SUN, SYBR gold, SYBR green, SYBR Safe, SYTO-9, SYTO-64, SYTO-82, TAMRA, TET, TEXAS RED, thiazole orange, TOTO-1, VIC, Yakima Yellow, or YOYO-1.

In some embodiments, the quencher group is selected black hole quencher 1 (BHQ1), BHQ2, BHQ3, DDQ-I, Dabcyl, Eclipse, Iowa Black FQ, QSY-7, DDQ-II, Iowa Black RQ, and QSY-21. In some embodiments, the quencher group comprises BHQ1. In some embodiments, BHQ1 comprises a moiety of 4′-(2-Nitro-4-toluyldiazo)-2′-methoxy-5′-methyl-azobenzene-4″-(N-ethyl)-N-ethyl. In some embodiments, the quencher group comprises BHQ2.

In some embodiments, the reporter group is located internally within a detection probe. In some embodiments, the reporter group is located at a 5′ or 3′ terminus of a detection probe.

In some embodiments, the quencher group is located internally within a detection probe. In some embodiments, the quencher group is located at a 5′ or 3′ terminus of a detection probe.

In some embodiments, each of the target-complementary specific sequences are independently conjugated at a 5′ or 3′ terminus with a Homo-Tag Assisted Non-Dimer System (HANDS) tag primer. In some embodiments, the HANDS tag primer is conjugated to the 5′ terminus of a target-complementary specific sequence. In some embodiments, the HANDS tag primer is conjugated to the 3′ terminus of a target-complementary specific sequence. In some embodiments, the HANDS tag primer comprises the nucleic acid sequence as set forth in SEQ ID NO: 11, or a sequence at least about 70%, at least about 75%, at least about 80%, at least about 85%, at least about 90%, at least about 95%, at least about 96%, at least about 97%, at least about 98%, at least about 99%, or more than 99% identical thereto.

In some embodiments, the method is performed in a single tube or well. In some embodiments, the method is performed in several tubes and/or wells.

In some embodiments, each of the two or more mediator probes independently comprise a chimera of D-DNA and L-DNA or D-RNA and L-RNA.

In some embodiments, each of the two or more mediator probes independently consist of D-DNA. In some embodiments, each of the two or more mediator probes independently consist of L-DNA. In some embodiments, each of the two or more mediator probes independently consist of D-RNA. In some embodiments, each of the two or more mediator probes independently consist of L-RNA.

In some embodiments, the non-complementary mediator sequence, the 5′ flap sequence, and/or the one or more detection probes comprise L-DNA or L-RNA residues. In some embodiments, the non-complementary mediator sequence comprises L-DNA or L-RNA residues. In some embodiments, the 5-flap sequence comprises L-DNA or L-RNA residues. In some embodiments, the one or more detection probes comprise L-DNA or L-RNA residues.

In some embodiments, the non-complementary mediator sequence, the 5′ flap sequence, and/or the one or more detection probes consist of L-DNA or L-RNA residues. In some embodiments, the non-complementary mediator sequence consists of L-DNA or L-RNA residues. In some embodiments, the 5-flap sequence consists of L-DNA or L-RNA residues. In some embodiments, the one or more detection probes consist of L-DNA or L-RNA residues.

In some embodiments, the sample is a human-, mammal-, or animal-derived material. In some embodiments, the sample is a human-derived material. In some embodiments, the sample is selected from a nasal swab, nasopharyngeal swab, throat swab, blood, plasma, serum, urine, sputum, spinal fluid, cerebrospinal fluid, pleural fluid, nipple aspirate, lymph fluid, respiratory tract fluid, intestinal tract fluid, genitourinary tract fluid, tear fluid, saliva, breast milk, lymphatic system fluid, semen, ascitic fluid, tumor cyst fluid, amniotic fluid, tissue, biopsy, feces, endotracheal aspirate, bronchoalveolar lavage, conjunctival swab, corneal swab, genital ulcer swab, lesion swab, endocervical swab, cervical scraping, cervical smear, vaginal swab, vulval swab, or a combination thereof.

In some embodiments, the sample is an environmental sample. In some embodiments, the sample is selected from a material sample including but not limited to an air filter sample, food, manure, or an environmental sample, including but not limited to water (e.g., tap water, well water, ocean water, wastewater), soil, or a combination thereof.

In some embodiments, the methods of any preceding aspect is capable of detecting at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 256, or more than 256 target nucleic acid sequences in a sample.

In one aspect, provided herein is a method for determining presence and/or amount of one or more target nucleic acid sequences derived from a virus or a bacterium in a sample, comprising: (i) designing one or more target specific sequences which are complementary to a target binding site in the one or more target nucleic acid sequences; and (ii) conducting the method of any aspect or embodiment disclosed herein using two or more mediator probes comprising the one or more target specific sequences of step (i).

In one aspect, provided herein is a method for determining presence and/or amount of one or more target nucleic acid sequences derived from one or more infectious agents, comprising: (i) designing one or more target specific sequences which are complementary to a target binding site in the one or more target nucleic acid sequences; and (ii) conducting the method of any aspect or embodiment disclosed herein using two or more mediator probes comprising the one or more target specific sequences of step (i). In some embodiments, the one or more infectious agents comprise a virus. In some embodiments, the one or more infectious agents comprise a bacterium. In some embodiments, the one or more infectious agents are selected from a blood culture pathogen, a gastrointestinal pathogen, a central nervous system infectious agent, a respiratory agent, or a pneumonia-related pathogen.

In some embodiments, the virus is selected from Adenovirus, Bocavirus, SARS-COV-2, Cytomegalovirus, Dengue virus, Influenza virus A, Influenza virus B, Hepatitis virus, Herpes Simplex Virus (HSV), human immunodeficiency virus (HIV), human metapneumovirus (hMPV), Human Papillomavirus (HPV), Norovirus, Orthopoxvirus, Parainfluenza virus, Respiratory Syncytial Virus (RSV), Rhinovirus, or Varicella-zoster viruses.

In some embodiments, the bacterium is selected from Streptococcus pneumoniae, Streptococcus pyogenes, Mycoplasma pneumoniae, Mycoplasma genitalium, Haemophilus influenzae, Stephylococcus aureus, MRSA, Bordetella pertussis, Chlamydia pneumonia, Chlamydia trachomatis, Klebsiella pneumoniae, Bacillus Anthracis, Candida auris, Candida tropicalis, Candida albicans, Candida parapsilosiis, Candida glabrata, Candida kruseii, Trichomonas vaginalis, Neisseria gonorrhoeae, Clostridium difficile, Coxiella burnetii, Entercoccus, Escherichia coli, Pseudomonas aeruginosa, Francisella tularensis, Mycobacterium tuberculosis, Streptococci spp., Salmonella spp., Cryptococcus neoformans, Legionella pneumophila, or Yersinia pestis.

In some embodiments, one or more infectious agents cause an infection or disorder selected from meningitis, encephalitis, meningoencephalitis, Human campylobacteriosis, periodontal infection, genital infection, diarrheal disease, Leishmaniasis, Genital ulcer disease, keratoconjunctivitis, or Acute respiratory tract infection.

In one aspect, provided herein is a method for determining presence and/or amount of one or more target nucleic acid sequences derived from one or more blood culture pathogens, comprising: (i) designing one or more target specific sequences which are complementary to a target binding site in the one or more target nucleic acid sequences; and (ii) conducting the method of any aspect or embodiment disclosed herein using two or more mediator probes comprising the one or more target specific sequences of step (i). In some embodiments, the one or more blood culture pathogens comprise Streptococcus pyogenes.

In one aspect, provided herein is a method for determining presence and/or amount of one or more target nucleic acid sequences derived from one or more gastrointestinal pathogens, comprising: (i) designing one or more target specific sequences which are complementary to a target binding site in the one or more target nucleic acid sequences; and (ii) conducting the method of any aspect or embodiment disclosed herein using two or more mediator probes comprising the one or more target specific sequences of step (i). In some embodiments, the one or more gastrointesinal pathogens comprise Salmonella.

In one aspect, provided herein is a method for determining presence and/or amount of one or more target nucleic acid sequences derived from one or more central nervous system infectious agents, comprising: (i) designing one or more target specific sequences which are complementary to a target binding site in the one or more target nucleic acid sequences; and (ii) conducting the method of any aspect or embodiment disclosed herein using two or more mediator probes comprising the one or more target specific sequences of step (i). In some embodiments, the one or more central nervous system infectious agents comprise Cryptococcus neoformans. In some embodiments, the one or more central nervous system infectious agents cause a central nervous system infection. In some embodiments, the central nervous system infection comprises meningitis, meningoencephalitis, or encephalitis.

In one aspect, provided herein is a method for determining presence and/or amount of one or more target nucleic acid sequences derived from one or more respiratory—and/or pneumonia-related pathogens, comprising: (i) designing one or more target specific sequences which are complementary to a target binding site in the one or more target nucleic acid sequences; and (ii) conducting the method of any aspect or embodiment disclosed herein using two or more mediator probes comprising the one or more target specific sequences of step (i). In some embodiments, the one or more respiratory—and/or pneumonia-related pathogens comprise Legionella pneumophila.

In one aspect, provided herein is a method for determining presence and/or amount of one or more target nucleic acid sequences derived from an environmental sample or material sample, comprising: (i) designing one or more target specific sequences which are complementary to a target binding site in the one or more target nucleic acid sequences; and (ii) conducting the method of any aspect or embodiment disclosed herein using two or more mediator probes comprising the one or more target specific sequences of step (i). In some embodiments, the material sample is selected from an air filter sample, food, manure. In some embodiments, the environmental sample is selected from tap water, well water, ocean water, wastewater, soil, or a combination thereof.

In one aspect, provided herein is a method for determining presence of a single-nucleotide polymorphism (SNP) in one or more target nucleic acid sequences in a sample, comprising: (i) designing one or more target specific sequences to a target binding site comprising a potential SNP in the one or more target nucleic acid sequences; and (ii) conducting the method of any aspect or embodiment disclosed herein using two or more mediator probes comprising the one or more target specific sequences of step (i).

In some embodiments, the one or more target specific sequences, or a fragment thereof, are complementary to the target binding site.

In one aspect, provided herein is a method for determining presence of a single-nucleotide polymorphisms (SNP) in one or more target nucleic acid sequences in a sample, comprising: (i) designing one or more target specific sequences which are complementary to a target binding site comprising a potential SNP in the one or more target nucleic acid sequences; and (ii) conducting the method of any aspect or embodiment disclosed herein using two or more mediator probes comprising the one or more target specific sequences of step (i).

In some embodiments, the one or more target specific sequences comprise a minor groove binder (MGB) moiety. An MGB moiety is a molecule that binds within the minor groove of double-stranded DNA. Suitable MGBs are described in the literature. See, e.g., U.S. Pat. No. 5,801,155; Wemmer, D. E., and Dervan P. B., Current Opinion in Structural Biology, 7:355-361 (1997); Walker, W. L., Kopka, J. L. and Goodsell, D. S., Biopolymers, 44:323-334 (1997); Zimmer, C.& Wahnert, U. Prog. Biophys. Molec. Bio. 47:31-112 (1986) and Reddy, B. S. P., Dondhi, S. M., and Lown, J. W., Pharmacol. Therap., 84:1-111 (1999) (the disclosures of which are herein incorporated by reference in their entireties for all purposes). In some embodiments, the MGB moiety comprises a polyamide. In some embodiments, the polyamide comprises a heterocyclic polyamide (comprising, e.g., pyrrole and/or imidazole groups). In some embodiments, the MGB moiety is a small molecule selected from netropsin, distamycin, pentamidine, berenil, or doxorubicin.

In some embodiments, the mediator probes and or the detection probes comprise one or more nucleotide modifications selected from Locked Nucleic Acid (LNA), Peptide Nucleic Acid PNA), Bridged Nucleic Acid (BNA), 2′-O alkyl substitution, L-enantiomeric nucleotide, or a combination thereof. In some embodiments, the one or more modified nucleotides comprise a 5-methylcytosine or 5-hydroxymethylcytosine. In some embodiments, the mediator probes and or the detection probes comprise one or more non-natural nucleotides. In some embodiments, the one or more non-natural nucleotides are selected from deoxyhypoxanthine, inosine, 1-(2′-deoxy-β-D-ribofuranosyl)-3-nitropyrrole, or 5-nitroindole.

In some embodiments, the determination of one or more SNP(s) using the methods of any aspect or embodiments described herein is associated with a disease or disorder selected from acute myeloid leukemia (AML), myelodysplastic syndrome, aggressive systemic Mastocytosi, B-cell chronic lymphocytic leukemia (CLL), bladder cancer, breast cancer, chromosome abnormalities, chronic myeloid leukemia (CML), Alzheimer's disease, Parkinson's disease, Celiac disease, Alpha-1 antitrypsin deficiency, primary Dystonia, Factor IX deficiency, Gaucher Disease Type 1, Glucose-6-Phosphate Dehydrogenase deficiency, hereditary Hemochromatosis, hereditary Thrombophilia, colorectal cancer, COVID-19, cystic fibrosis, endometrial cancer, follicular lymphoma, Fragile X, a hematologic malignancy, lung cancer, small cell lung cancer (SCLC), non-small cell lung cancer (NSCLC), colon cancer, melanoma, Lynch syndrome, multiple myeloma, acute lymphoblastic leukemia, myeloproliferative disease, polycythemia vera, ovarian cancer, prostate cancer, Severe Combined Immunodeficiency Disorder (SCID), Spinal muscular atrophy (SMA), urothelial cancer, meningitis, Influenza, or a combination thereof.

In some embodiments, the methods are performed on a sample derived from a subject in need thereof. In some embodiments, the subject is an embryo or a fetus. In some embodiments, the subject is a newborn. In some embodiments, the subject is a child or adolescent. In some embodiments, the subject is an adult. In some embodiments, the subject is a human.

### Kits

In one aspect, provided herein is a kit for use with a method of any aspect or embodiment described herein. In some embodiments, the kit includes two or more mediator probes, an enzyme having 5′ exonuclease activity and/or a nucleic acid polymerase, and one or more detection probes. In some embodiments, L-DNA is included in the kit. In some embodiments, the kits comprise, in suitable container means, reagents including dyes, labels, standards, detection devices, and instructions for using the same.

In one aspect, the kit is used for detecting one or more target nucleic acid sequences in a sample by PCR assay.

In some embodiments, the reagents may further comprise suitably aliquoted compositions. In some embodiments, the kits comprise individual reagents or mixed reagents, in one or more separate containers. In some embodiments, the components of the kits are packaged either in aqueous media or in lyophilized form.

In some embodiments, the containers of the kits include at least one vial, test tube, flask, bottle, syringe or other container means, into which the reagents may be placed, or preferably, suitably aliquoted. In some embodiments, the kits of the present disclosure will also typically include a means for containing the reagent containers in close confinement for commercial sale. In some embodiments, the containers include injection or blow-molded plastic containers into which the desired vials are retained.

In some embodiments, L-DNA is not included in the kit.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Exponentially Scalable PCR for Multiplex Detection of Pathogens in a Single Tube

Recent work that has shown that multiplex capabilities can be expanded beyond the traditional number of fluorescence channels is extended by reagent designs that associate the PCR detection of a target with a signature sensing element comprised of a unique melt temperature in a particular fluorophore channel. The example herein demonstrates that a simple method to create simultaneous sensing signature elements, or “bits”, for each target of interest can be used to create multi-bit combinatoric codes, uniquely identifying many targets but with fewer sensing elements. The method, referred to as multi-bit PCR, enables the unique identification of an exponential number of unique targets, for example including but not limited to in a single tube. The scalability of this PCR product identification is developed and characterized based on simultaneous multi-bit coding and applies the approach to a syndromic panel of clinically relevant respiratory pathogens consisting of 16 unique targets. The multiplexing approach is extended by an order of magnitude through development of single-tube reagents for a comprehensive diagnostic panel consisting of >100 pathogens of interest spanning multiple infection types. This approach creates a new implementation paradigm to enable multi-target detection in highly multiplexed PCR within, for example a single tube on standard PCR instrumentation.

Disclosed herein is a multiplex panel to differentiate amongst target nucleic acid sequences, for example respiratory pathogens with similar symptoms. To enable the multi-bit PCR methods disclosed herein on standard PCR instrumentation, a novel encoding method (i.e., referred to as a “combinatoric code” herein) is developed to uniquely identify one PCR target among the many possible. Because of the potential applicability to many multiplex PCR reactions, the limits of the encoding method are further tested to determine the upper limit on the number of unique targets detectable with this approach.

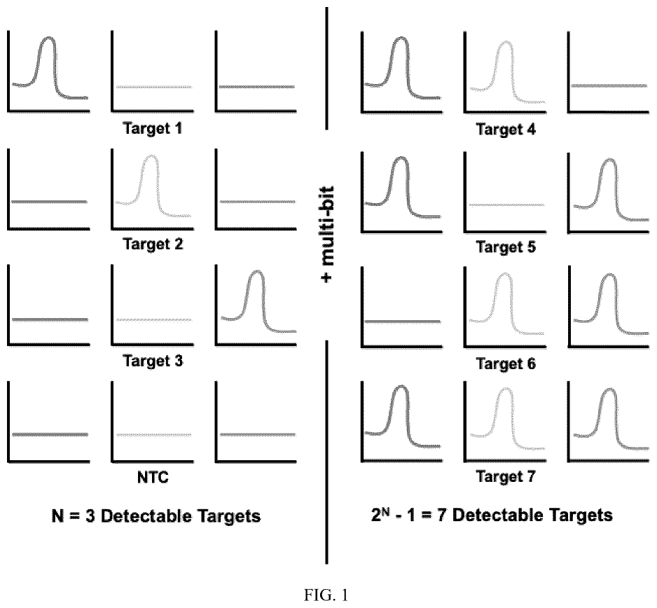

The high analytical sensitivity and high specificity of PCR has made it the gold standard for determining the presence of a particular target nucleic acid sequence, for example a nucleic acid sequence derived from an infectious pathogen. Each additional PCR test that can be added to a reaction increases the utility of this gold standard approach. Assuming one could combine as many individual PCR reactions as desired into, e.g., a single tube, state-of-the-art PCR instruments and detection reagents limit the maximum number of positive PCR reactions to one reaction for each of the instrument's fluorescence channels. A general method is disclosed herein which enables highly multiplexed PCR reactions in, e.g., a single tube, for testing hundreds of targets at once. To do this, recent reagent designs that have been shown to improve multiplex capabilities beyond the traditional number of fluorescence channels are modified as disclosed herein. These reagent designs associate the PCR detection of a target with a unique signature element that corresponds to one melt temperature in a fluorophore channel. The PCR detection of a target and the signature event has a 1-to-1 relationship, limiting the number of discernable targets to the number of unique fluorophore-melt temperature signature elements. As such, the number of possible targets scales linearly with the number of identifiable sensing elements. Further scaling is not possible without increasing either the number of fluorescence channels and/or the number of resolvable melt peaks within each channel. This disclosure demonstrates a simple method to combine signature elements into multi-bit combinatoric codes for each target of interest which reduces the required number of sensing elements. This simplifies the assay implementation and the required PCR instrumentation. This method, referred to as multi-bit PCR, overcomes the remaining technical identification challenge by enabling the unique identification of an exponential number of unique targets in a single tube. FIG. 1 shows detection of seven targets by combining three unique sensing elements.

The feasibility and clinical utility of the multi-bit multiplexing strategies are explored herein. Syndromic respiratory panels, for example, are an excellent application where many infectious pathogens produce similar clinical symptoms, and, therefore, initial tests are designed for testing a panel of clinically relevant respiratory pathogens consisting of ˜16 unique targets in a single tube. Performance and limitations of the assay are measured, including theoretical and practical multiplexing bounds. Following characterization, the diagnostic panel is adapted to include ˜100 pathogen targets which are screened for in the clinic.

### Develop and Validate Reagent Designs for Multi-Bit PCR

The scalability of PCR product identification is developed and characterized based on simultaneous multi-bit coding. Starting designs are based on previously vetted PCR reagents for a panel of 16 respiratory pathogens and on recently published “mediator probe” designs that provide the interface between the target detected during PCR and a “reporter” molecular beacon structure detected by traditional PCR instrumentation. Reagents are validated in simplified test designs, applied to determine the maximum number of simultaneous signature elements per target, and then incorporated to test the multiplex design for the 16 pathogens. Overall performance is evaluated based on the trade-offs between the analytical sensitivity of each target determined by multiplex detection versus singleplex detection.

### Expand the Number of Multiplexed Targets to Achieve Detection of >100 Pathogens

Core design principles are applied to develop single-tube reagents for a comprehensive diagnostic panel consisting of >100 pathogens of interest spanning multiple infection types. The reagent and assay design features are identified and applied at a scale for exploring the practical feasibility of the multi-bit approach. Targets are tested in multiplex reactions, and the qualitative analytical accuracy is compared to the gold standard traditional PCR which relies on separate tube reactions for each standard.

This approach enables multi-target detection in highly multiplexed PCR, enabling identification of comprehensive panels focusing on pathogens as well as many other clinically relevant multiplex reactions.

### BACKGROUND

### Multiplex PCR Challenges

Polymerase chain reaction (PCR) is one of the most widely used diagnostic technologies due to its unparalleled sensitivity and specificity. Multiplex PCR enables simultaneous detection of multiple target sequences, broadening the diagnostic scope and conserving time and resources. Multiplex PCR technologies have remarkable clinical usefulness as demonstrated by the existence of several diagnostic panels and the continued development for lab-developed tests (LDTs).

Examples of these include BIOFIRE® FILMARRAY® (bioMerieux), QIAstat-Dx (Qiagen), and cobas eplex (Roche) which are used in syndromic diagnostic applications such as respiratory pathogen panels. However, while clinicians desire to use multiplex PCR panels routinely, the highly multiplexed PCR panels are often under-utilized in the clinic because of high costs. The expensive nature of these tests can be attributed to the complex approaches for overcoming technical challenges inherent to multiplex PCR, such as specialized instrumentation and microfluidic separation of PCR reactions. Two notable challenges increase the difficulty of high order multiplexing. First, the limited fluorescent color channels for identifying targets by fluorescent means (e.g., hydrolysis probes) constrains the number of unique targets identifiable in a single multiplex reaction. Second, as PCR multiplexing increases, the risk of interactions between the additional primer oligonucleotides (i.e., primer-dimers) increases, resulting in lower PCR efficiency and high false positive rates.

Key modifications are implemented herein to address these technical limitations of multiplex PCR reactions. Melt analysis is commonly used to overcome fluorescence channel limitations, such as in BIOFIRE® FILMARRAY® panels. This approach generates melt temperature signals characteristic of PCR amplicon length. However, amplicon-dependent melt analysis is limited by amplicon size and specificity requirements of PCR, reducing the number of discernable targets in an individual multiplex reaction. To overcome these limitations, mediator probe and melt analysis have been combined in the methods described herein, to uniquely identify, in some non-limiting embodiments described herein, the outcome of up to 62 individual PCR reactions with low primer dimer formation. In this design, mediator probes that translate a PCR extension event to a molecular beacon melting event expands the unique identification capabilities of standard PCR instrumentation, that is it expands the “sensing” modalities of the instrument. Also, in some non-limiting embodiments described herein, the risk of primer-dimer formation has also been reduced by including the Homo-Tag Assisted Non-Dimer System (HANDS).

The methods disclosed herein adapt the melt signature modality to a combinatoric (e.g., binary) encoding algorithm to exponentially increase the number of unique identifiers detectable by a traditional PCR instrument. The adaptation, referred to here as multi-bit PCR, enables up to a theoretical 262 unique identifiers for target identification in a single tube and the method can be implemented on any PCR instrument with melt analysis capabilities. In some non-limiting embodiments, the method uniquely codes for up to 28 targets using simpler instrument and reagent designs-aiming for a minimum of two color channels and four melt temperatures in each. The scalability of the multi-bit PCR methods as disclosed herein is exemplified by extending this to four color channels each with four melt temperatures to investigate.

### Taq Polymerase Hydrolysis Activity and Molecular Beacon Melt Analysis Provide Signature Elements for Expanded Detection in a Single Fluorescence Channel

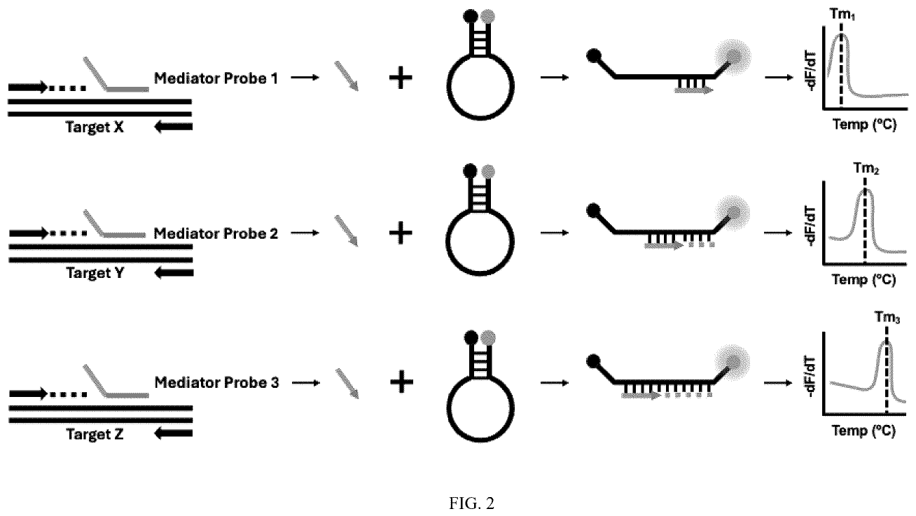

A key limitation of traditional multiplex PCR is the limited number of fluorescence channels available for monitoring PCR target detection. Typical multiplex PCR implementations use hydrolysis probes labeled with fluorescent reporters that associate one target with one reporter, restricting the number of detectable targets to the number of independent fluorescence channels. Although not in a multiplex design, universal molecular beacon reporters have been demonstrated to enable the detection of multiple targets within the same fluorescence channel to address this challenge. In this design, the hydrolytic activity of Taq polymerase is leveraged to cleave specific 5′-flap sequences appended to PCR hydrolysis probes, called “mediator probes”. This is the same hydrolytic activity responsible for fluorophore release for standard hydrolysis probes (e.g., TaqMan probes). The cleaved 5′-flap sequence, called a “mediator primer,” is complementary to a specific capture sequence, e.g., a loop region of a fluorescently labeled molecular beacon, that then produces signal upon polymerase extension. Melt analysis was incorporated to switch the outcome of PCR extension from a color to a melt peak within a color channel, overcoming the low-resolution length restrictions inherent in amplicon-melt methods. This expands the number of PCR instrument sensing modalities by adding multiple characteristic melt temperatures (Tms) to each fluorescence channel. The molecular mechanism is illustrated in FIG. 2. After cleavage, mediator primers (FIG. 2) are designed to specifically bind to one of many sites within the loop region of the molecular beacon which, following polymerase extension of the flap sequence, produces specific length double-stranded DNA sequences with distinct Tms. Adding multiple fluorogenic molecular beacons allowed up to 62-plex PCR through fluorescence and Tm signature. Importantly, the integrity of the high order multiplexed PCR was preserved by minimizing primer dimer interactions with the HANDS technology, described below.

### Homo-Tag Labeled Primer Designs Minimizes Primer-Dimerization Risk

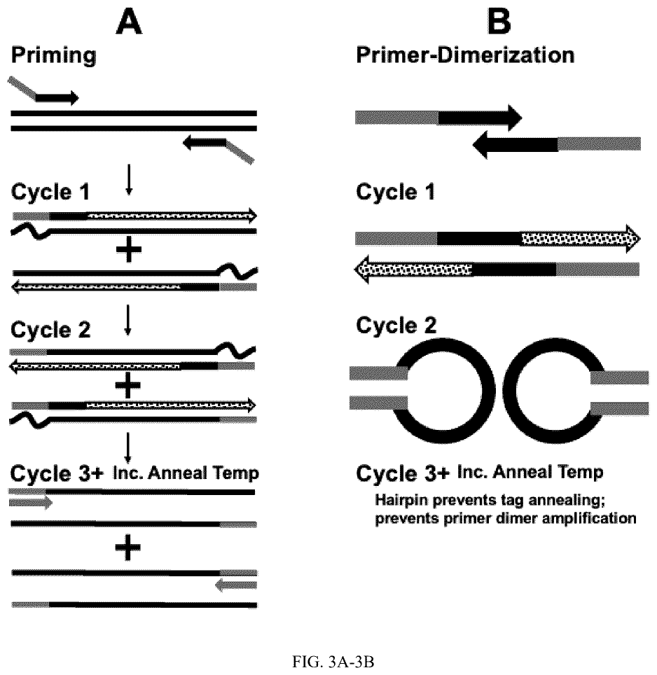

Reduced PCR efficiency due to primer dimer interactions is a major risk when multiplexing PCR. The likelihood of primer dimer occurrence increases with additional primer sets, adding complexity to multiplex PCR design. To reduce primer dimerization, the Homo-Tag Assisted Non-Dimer System (HANDS) was developed (FIG. 3). Fundamentally, HANDS achieves dimer reduction by including universal “tag” sequence 5′ tails on each specific primer set, which are designed with the same sequence for both strand senses (i.e., homo-tag). PCR cycling is initiated with the inner (3′) specific primer sequence, which recognizes the target of interest and inserts the 5′ tag sequence into the amplicon. After a few cycles, annealing temperature is increased to facilitate only amplification by the universal primer tag region. In the event of primer dimer extension, the homo-tag complement is incorporated into the undesired primer amplicon. The homo-tag and complement sequence then form a hairpin, preventing further amplification of the primer-dimer amplicon and successfully limiting the reduction of PCR efficiency.

As such, the HANDS technology enables highly multiplexed PCR reactions and has been demonstrated in 62-plex when used with the fluorescence channel-melt temperature method. The fluorescence-melt design scales linearly with the number of identifiable targets. Therefore, as targets are increased, the number of molecular beacons necessary increases quickly-increasing costs, molecular design complexity, and the need for more instrument fluorescence channels. To overcome these limitations, multi-bit PCR employs multiple mediator probes for an individual target sequence and a combinatoric identification scheme.

Fluorescence Channel and Melt Temperature Elements are Combined into Unique Binary Codes to Increase the Number of Identifiable Targets in a Multiplexed Reaction

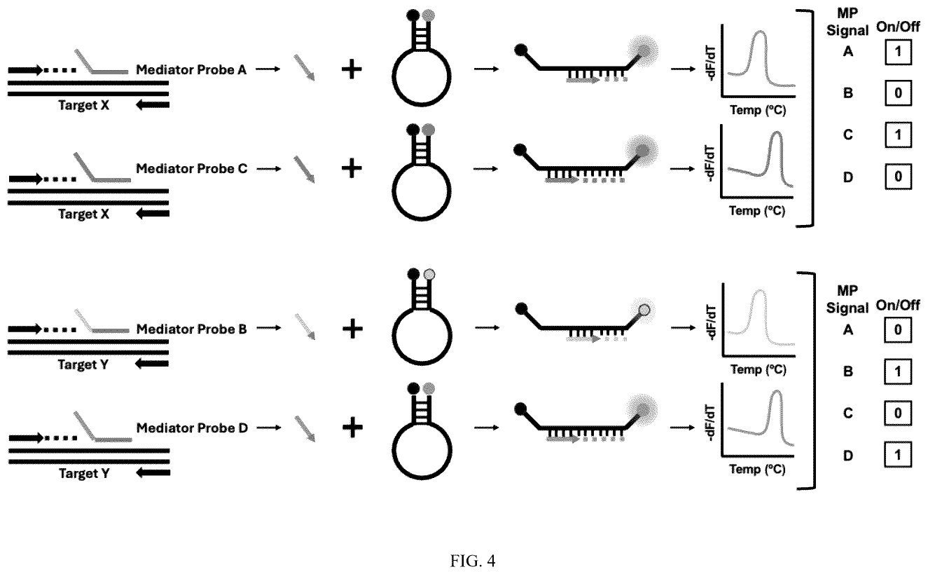

By leveraging the established HANDS and fluorescence-Tm signature approaches, the methods disclosed herein exponentially increase the number of uniquely detectable targets in a single tube. The method associates a positive PCR reaction with combinations composed of multiple simultaneous Tm and fluorescence signature elements. The signature combination provides a binary-based unique identifier code that is selected from one of 2N possibilities, derived from the signal or lack thereof (i.e., 1 or 0) from N different fluorescence-Tm signature elements. This approach is achievable simply by including multiple mediator probes all designed to bind on a target nucleic acid sequence at, for example including but not limited to a single target binding site. For a given target nucleic acid sequence, the design of bi-functional multiple mediator probes is straight-forward. Each of the multiple mediator probes contains the same target binding site (referred to herein as the complementary “target specific sequence”), enabling the probe detection function. Importantly, each is also concatenated with one of the available mediator primer sequences (referred to herein as the non-complementary “mediator sequence”), enabling the instrument sensing function. When a PCR target is present, the collective response corresponds to a unique identifier combinatoric code using the instrument's fluorescence-Tm sensing capabilities (FIG. 4). As the PCR reaction progresses, the exponential amplification of amplicons provides sufficient binding sites for the additional mediator probes necessary to achieve the identifier code combinations. The result of this approach is a single tube PCR reaction (multi-bit PCR) capable of theoretically detecting 2N-1 PCR targets, where N is equal to the number of signature sensing elements (i.e., total number of melt temperatures across all fluorophore-specific channels).

Disclosed herein in non-limiting embodiments is the development and validation of reagents to achieve high order multi-bit PCR, which establish the principle mechanisms for generalized use of the technology, and demonstrate the strategy with a 16-plex respiratory panel and a >100-plex comprehensive diagnostic panel. Provided herein is a generalizable, exponentially scalable PCR approach applicable to many other PCR-based diagnostic assays where, for example, many pathogens result in similar symptom presentation or where a large number of specific targets can be ruled out.

The scaling of multi-bit PCR methods disclosed herein are implemented by using multiple mediator probes per target to generate multi-bit combinatoric codes as unique PCR target identifiers (FIG. 4). The number of signature elements available for unique encoding is equivalent to the total number of melt temperatures across all fluorescence channels. For example, if the outcome of a multiplex reaction is measured using four melt temperatures in each of two fluorescence channels, this results in 8 bits for binary encoding or 28 combinations for identification, that is unique identification of up to 256 targets.

There are two major technical innovations incorporated in this disclosure: (1) inclusion of multiple mediator probes for each individual target nucleic acid sequence and (2) addition of a combinatoric algorithm for binary encoding and identification of targets. Both innovations lead to high order PCR multiplexing achievable on a standard PCR instrument, for example including but not limited to in a single tube. An additional benefit is that multi-bit PCR attains high order multiplexing even with simpler PCR machines that have limited fluorescence channels or lower resolution melt analysis. The multiple mediator probe approach provides exponential scaling due to the combinatoric nature of the binary coding algorithm, surpassing the linear scaling limitations of previous implementations. Since multi-bit PCR achieves exponential target identification, more unique targets are testable in a single tube while requiring less reagents. Therefore, multi-bit PCR is less costly and easier to implement than the high order linearly scaled multiplexing approaches. While the method is developed for a 16-plex respiratory panel and a >100-plex comprehensive infectious diagnostic panel, the innovations outlined in this approach are not tied to any particular sequence(s). Therefore, a major strength of multi-bit PCR is the adaptability to any PCR application where multiplex capabilities are desirable.

### Experimental Design

The description herein allows the unique identification of a large number of PCR targets in a single tube multiplex reaction with standard PCR instrumentation. The reagents underlying the methods disclosed herein are developed and validated with a proof-of-concept syndromic respiratory panel and to test the limits of the exponential target detection capabilities of the method. Tables 1 and 3-4 are provided to outline the objectives with corresponding experimental plans.

### Develop Reagent Designs for Multi-Bit PCR

A critical component to enabling the multi-bit PCR is the capacity to incorporate multiple hydrolysis mediator probes into the detection of a single target sequence, and it is essential to identify the mechanism(s) to achieve this feature without major impacts on the analytical sensitivity of PCR. By implementing the generation of two simultaneous sensing elements for a single PCR extension event, the practicality of the multi-bit PCR methods disclosed herein are demonstrated for real clinical use cases (i.e., respiratory panel) while achieving an iterative step toward identifying a large number of PCR targets in a single tube. A 16-plex PCR respiratory detection platform is achieved that only requires two fluorophore channels.

Respiratory panel targets are informed by infectious disease clinical considerations and current commercially available panels. In some non-limiting embodiments, the respiratory pathogens are selected from rhinovirus, influenza virus A (flu A), influenza virus B (flu B), respiratory syncytial virus (RSV), SARS-COV-2, adenovirus, parainfluenza virus, human metapneumovirus (hMPV), bocavirus, Streptococcus pneumoniae, Mycoplasma pneumoniae, Haemophilus influenzae, Staphylococcus aureus (including MRSA), Bordetella pertussis, Chlamydia pneumoniae, Klebsiella pneumoniae, or a combination thereof.

TABLE 1
Overview of Objectives

| Objective | | Experimental Plan | Outcomes |
|---|---|---|---|
| 1 | Validate | Perform PCR extension and melt | Eight (8) unique mediator |
| | reagents for | analysis with synthesized mediator | primers produce specific |
| | mediator | primers and molecular beacons; | Tms across two (2) |
| | sequences and | measure repeatable melt | molecular beacons w/ |
| | molecular | temperatures (Tms) and Tm | standard deviation <1° C. |
| | beacons. | variances for each pair | |
| 2 | Establish | Design mediator probes | Successful detection of |
| | mechanism for | incorporating mediator primer and | synthetic target by melt |
| | incorporating | beacon pairs established in 1.1; test | analysis to <100 target |
| | multiple | probe number limits per synthetic | DNA copies; capacity for ≥4 |
| | mediator probes | target sequence by PCR and melt | mediator probes at single |
| | for a single | analysis; determine analytical | binding site for a given |
| | target sequence. | sensitivity limits with increased # | target sequence |
| | | of mediator probes | |
| 3 | Develop highly | Design and perform multi-bit PCR | Successful detection by |
| | multiplexed | with two dimensions (two | multi-bit melt analysis for |
| | respiratory panel | molecular beacons) for common | sixteen (16) respiratory |
| | with multi-bit | respiratory targets (w/reagents | pathogens; LODs ≤100 |
| | PCR. | from 1.1); test limits of detection | target copies for all 16 |
| | | (LODs) and specificity with | targets; >95% specificity |
| | | synthetic oligonucleotide targets | |

| Milestone | Detection of 16 targets in a single PCR reaction with only two |
|---|---|
| | fluorophore color channels. |

### Reagents for Mediator Sequences and Molecular Beacons

The melt system is experimentally isolated by directly adding the mediator primer sequences (as if they were cleaved from the mediator probe) to PCR master mix containing the molecular beacon(s). Mediator primer sequences are used that were previously designed to not interact with any known organism sequences. Mediator primers for this study are shown in Table 2.

TABLE 2
Oligonucleotide mediator primers (P) and paired molecular beacon (MB) reporters. Each
beacon is paired with four mediator primers which, following mediator primer extension,
generates four different double-stranded product each with different lengths. The
difference in length results in four distinct melt temperatures (Tms)

| Oligo | Sequence (5′ > 3′) | Product Length |
|---|---|---|
| MB1-P1 | CGCTCTCCGTGCCCACTC (SEQ ID NO: 1) | 23 |
| MB1-P2 | CAGCGCTCTCCGTGCCCA (SEQ ID NO: 2) | 26 |
| MB1-P3 | ACACTGTCCAGCGCTCTC (SEQ ID NO: 3) | 34 |
| MB1-P4 | GGTCCACACTGTCCAGCG (SEQ ID NO: 4) | 39 |
| MB1 | /FAM/CGGCGGAGTGGGCACGGAGAGCGCTGGACAGTGT | — |
| | GGACCCACGTCTCGCAGCAGGCCGCCG/BHQ1/ | |
| | (SEQ ID NO: 5) | |
| MB2-P1 | ACACTTCTTTTTGCTCG (SEQ ID NO: 6) | 17 |
| MB2-P2 | TCTCACACTTCTTTTTG (SEQ ID NO: 7) | 21 |
| MB2-P3 | CACCTCTCACACTTCTT (SEQ ID NO: 8) | 25 |
| MB2-P4 | ATCACACCTCTCACACT (SEQ ID NO: 9) | 29 |
| MB2 | /ROX/CGAGCAAAAAGAAGTGTGAGAGGTGTGATGAGCTCG/BHQ2/ | — |
| | (SEQ ID NO: 10) | |

All studies, including these isolated validation studies, use SensiFAST Probe No-ROX Kit (Thomas Scientific, Cat #C755H90). PCR extension is performed at 35° C. for 30 minutes to form the mediator primer and molecular beacon specific double-stranded sequence. The specific double-stranded sequences are analyzed by melt analysis, a standard feature in modern real-time PCR instruments, to determine characteristic melt temperatures (Tms) and their respective variances. Following a 95° C. for 1 min and 45° C. for 1 min melt preparation, melt analysis is performed from 45° C. to 95° C. at a ramp rate of 1° C. per 5 seconds. Extension and melt analysis is performed on a Rotor-Gene Q PCR (Qiagen). Optimization includes temperature and duration of the extension step, concentration of the mediator primers and molecular beacons, and location and length of the mediator primers with respect to the beacons. To ensure downstream developmental success, characteristic Tms with standard deviations <1° C. are achieved. Sufficient signature elements for achieving the remaining objectives are established with eight unique mediator primers that span two molecular beacons (four primers/beacon) (Table 2).

### Mechanism for Incorporating Multiple Mediator Probes for a Single Target Sequence

During traditional PCR, the copy number of viable hydrolysis probe binding sites exponentially increases. Therefore, the same mediator probe binding sequence is used as the basis for initiating 5′-flap cleavage of many different mediator primer sequences simultaneously. The effect on system analytical performance is determined by using three representative target sequences with an increasing number of mediator probes. Targets and primer/probe reagents are selected from previously validated assays. Mediator probe studies are conducted with reagents and instruments outlined herein. PCR cycling conditions is as follows: 95° C. for 5 min; 45 cycles of 95° C. for 15 sec and 60° C. for 1 min; extension at 35° C. for 30 min; melt preparation at 95° C. for 1 min then 45° C. for 1 min; melt analysis from 45° C. to 95° C. at 1° C. per 5 sec. Mediator probe concentrations are optimized by evaluating concentration ranges from 10 nM to 500 nM. Successful detection of target down to <100 copies per reaction with at least 4 mediator probes in the assay for a single target binding site.

Highly Multiplexed Respiratory Panel with Multi-Bit PCR

A 16-plex PCR syndromic respiratory panel is developed while using a fraction of the potential for multi-bit PCR. Using the two molecular beacons and eight mediator primers, 28 (256) unique binary identifiers are available to be assigned to 16 common respiratory pathogens. Synthetic oligonucleotide sequences representative of the 16 respiratory pathogens are tested with the multi-bit PCR method disclosed herein. Analytical performance is characterized by performing limit of detection (LOD) studies across all 16 sample targets while observing occurrence of false positivity to determine analytical specificity. In some non-limiting embodiments, HANDS tag universal primers are appended to the end of target specific primers to minimize the potential risk for primer-dimers in 16-plex. The HANDS universal tag sequence 5′-GCAAGCCCTCACGTAGCGAA-3′ (SEQ ID NO: 11) is used. To accommodate the HANDS approach, PCR cycling conditions are modified as follows: 95° C. for 5 min; 4 cycles of 95° C. for 15 sec and 60° C. for 1 min; 45 cycles of 95° C. for 15 sec and 65° C. for 1 min. The remaining PCR cycling conditions match cycling embodiments disclosed herein. Studies are performed with concentrations and materials established herein.

### Expanding the Number of Multiplexed Targets to Achieve Detection of >100 Pathogens

The multi-bit PCR is capable of uniquely identify a number of targets well beyond practical use. The effectiveness and promise of multi-bit PCR increases the scale of achievable multiplex targets while remaining within practical testable limits. To achieve this, the multi-bit PCR is extended to incorporate double the mediator primers and the molecular beacons disclosed herein, which squares the number of detectable targets (from 28 to 216). While 216 unique identifiers surpass conventional, practical testability, a >100-plex comprehensive diagnostic panel is designed and tested to show the potential of multi-bit PCR. Success demonstrates the proof-of-concept for multi-bit PCR to achieve single tube comprehensive infectious disease diagnostic testing.

Comprehensive infectious disease panel targets is informed by infectious disease clinical considerations and current commercially available panels. The pathogens include 16 respiratory targets as disclosed herein and is extended to include targets across various sample types that include but are not limited to: blood culture pathogens (e.g., Streptococcus pyogenes), gastrointestinal pathogens (e.g., Salmonella), central nervous system infectious agents (meningitis/encephalitis) (e.g., Cryptococcus neoformans), and additional respiratory/pneumonia-related pathogens (e.g., Legionella pneumophila).

TABLE 3
Overview

| Objective | | Experimental Plan | Outcomes |
|---|---|---|---|
| 1 | Validate reagents | Extend to 8 more mediator primers | Sixteen (16) unique |
| | to extend multi- | and 2 more molecular beacons; | mediator primers produce |
| | bit PCR for | perform PCR extension and melt | specific melt temperatures |
| | higher order | analysis to measure Tms and Tm | across four (4) molecular |
| | multiplexing. | variances for each pair | beacons w/standard |
| | | | deviation <1° C. |
| 2 | Achieve single | Design and perform multi-bit PCR | Successful detection of >100 |
| | tube | with four dimensions (four | unique target types in |
| | comprehensive | molecular beacons) for commonly | a single PCR reaction; |
| | diagnostic panel. | screened diagnostic panels; | LODs ≤500 target copies |
| | | determine analytical performance | for all targets; >90% |
| | | of highly multiplexed PCR | specificity |

| Milestone | Detection of >100 targets in a single PCR reaction with four |
|---|---|
| | fluorophore color channels monitored. |

### Reagents to Extend Multi-Bit PCR for Higher Order Multiplexing

Eight more mediator primers and two more molecular beacons are added and validated in the multi-bit PCR system to achieve binary encoding of up to 216 targets. New mediator primers and molecular beacons are screened in isolated studies to ensure Tm uniqueness and minimal Tm variance (<1° C.). Study conditions are performed as outlined in some study embodiments disclosed herein. All sixteen mediator primers and four molecular beacons are tested in varied combinations to verify absence of cross-reactivity between unintended pairs.

### Single Tube Comprehensive Diagnostic Panel

A single tube comprehensive diagnostic PCR panel has the potential to be a one-for-all diagnostic assay. By expanding the dimensions from two to four, multi-bit PCR exponentially scales to achieve significantly more unique signatures appliable to the comprehensive diagnostic PCR panel. Scalability allows detection of >100 target types in a single tube. Target types are based around commonly screened pathogens across multiple sample types and informed by infectious disease clinicians for practicality. Target types are designed spanning the 216 unique combinations to minimize signature overlap. Reagent concentrations, PCR cycling conditions, and materials are performed as outlined in conditions and materials of embodiments disclosed herein.

TABLE 4
Timeline

| Objective | | Year 1 | | | | Year 2 | | | |
|---|---|---|---|---|---|---|---|---|---|
| 1.1 | Reagents for mediator sequences and | x | | | | | | | |
| | molecular beacons. | | | | | | | | |
| 1.2 | Mechanism for multiple mediator probes for | x | x | | | | | | |
| | single target sequence. | | | | | | | | |
| 1.3 | Highly multiplexed respiratory panel with | | x | x | x | | | | |
| | multi-bit PCR. | | | | | | | | |
| 2.1 | Reagents for higher order multiplexing. | | | | x | x | x | x | |
| 2.2 | Single tube comprehensive diagnostic panel. | | | | | x | x | x | x |

### Example 2. High-Order Target Detection by Multi-Dimensional PCR

Proof-of-concept data is disclosed herein demonstrating performance of foundational reagents, their application to a practical pathogenic assay use case, and the extension of the technology to be coupled with other enzymatic assays for additional applications. The exemplary developments described herein largely focus on development of underlying technology (i.e., mediator probe PCR) to enable the Multi-Dimensional PCR platform technology disclosed herein. Additionally, developments were made to demonstrate the broader application base beyond traditional PCR toward simple, clinically relevant genotyping.

### Reagent Development

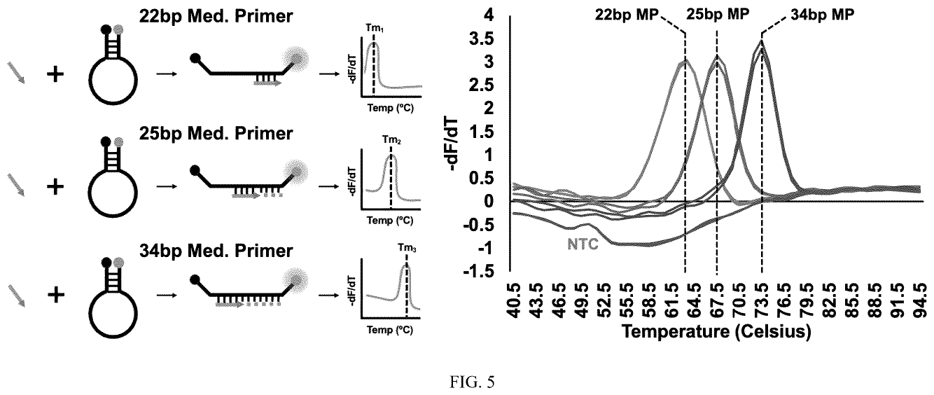

Assay reagents including the DNA polymerase, PCR master mix and components, initial molecular beacon reporters, and three unique, beacon specific mediator primers have been established for early validations of the multidimensional PCR approach (FIG. 5). Following polymerase extension and melt analysis, the initial molecular beacon reporter and three unique mediator primers generated three clearly discernable and distinct signals with minimal variability (n=3 samples per primer; NTC: no target control). These reagents were used for further preliminary investigations.

### Mediator Probe PCR Application Example: Treatment-Grouped Atypical Bacteria

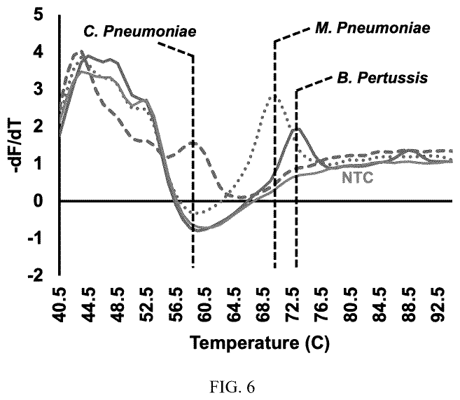

The multi-dimensional PCR approach is built upon a mediator probe PCR approach to enable the exponentially scalable multiplexing. To demonstrate the feasibility of mediator probe PCR as the foundation of the multiplex Multi-Dimensional PCR platform methods disclosed herein, a management-grouped pathogen detection panel was designed and evaluated on three atypical bacteria: C. pneumoniae, M. pneumoniae, and B. pertussis (FIG. 6). These studies were performed with synthetic gene blocks for C. pneumoniae and M. pneumoniae and an extraction of chemically inactivated B. pertussis. The multi-dimensional PCR approach was performed with one molecular beacon reporter to report for all three distinct targets. FIG. 6 shows that individual targets can be associated with a unique melt temperature-fluorescence signature. Here, three separate tubes containing individual targets were overlayed on the same graph to show their distinct mediator probe signatures and their relationship to each other. Each of the 3 bacterial targets was consistently detected by mediator probe PCR when tested individually on a single fluorescence channel.

### Broader Applications: Leveraging Multi-Dimensional PCR to Develop PCR-Based Pipeline for Single-Nucleotide Detection and Clinically Relevant Genotyping.

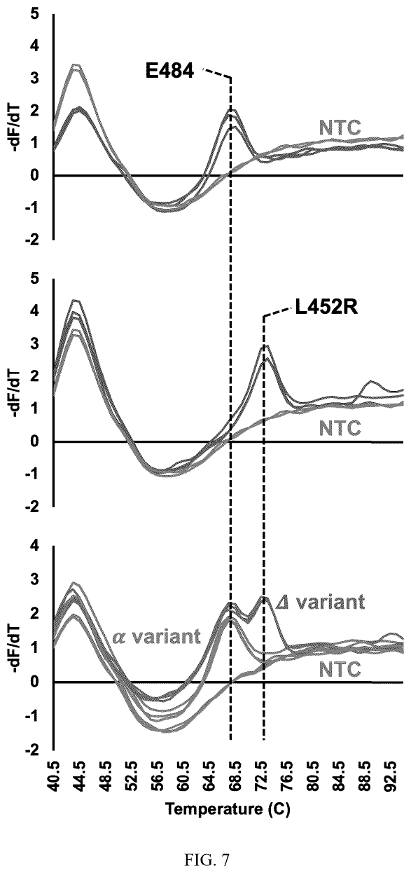

Preliminary investigations for the highly multiplexed PCR-based genotyping pipeline focused on the adaptation of established SARS-COV-2 variant-typing reagents (Nelson, et al. IRV. 2023) for the oligonucleotide ligation assay (OLA) coupled with mediator probe PCR. Reagent designs and experimental conditions were established and evaluated for the SNVs E484 and L452R, characteristic of Alpha and Delta variants, respectively. Initially, synthetic oligonucleotides containing the SNVs and their surrounding sequences were evaluated with OLA followed by the new mediator probe PCR to determine the signature fluorescence-melt temperature response (FIG. 7, Top and Middle Panel). To demonstrate clinical feasibility, previously sequenced SARS-COV-2 clinical samples (Alpha and Delta variants) were extracted and evaluated with the PCR-based genotyping methods disclosed herein, after performing a previous protocol (Nelson, et al. IRV. 2023) (FIG. 7, Bottom Panel). The OLA and mediator probe PCR resulted in consistent detection and identification of SNVs indicative of both Alpha (red) and Delta (blue) variants (n=3) with fluorescence-melt temperature signals matching their corresponding synthetic oligo responses.

These preliminary results demonstrate clinical potential of a SNV detection method as disclosed herein.

### Example 3. Highly Scalable, Combinatoric PCR Strategy Enables Augmented Panel-Based Multiplexing for Single Target Detection

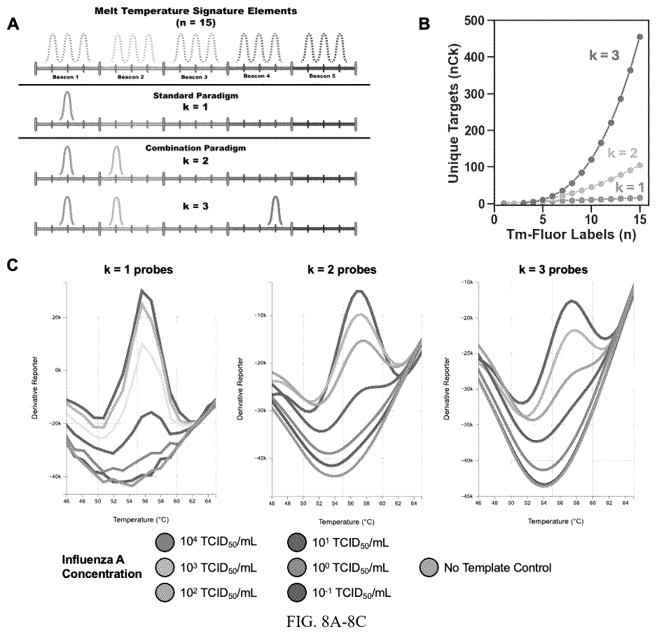

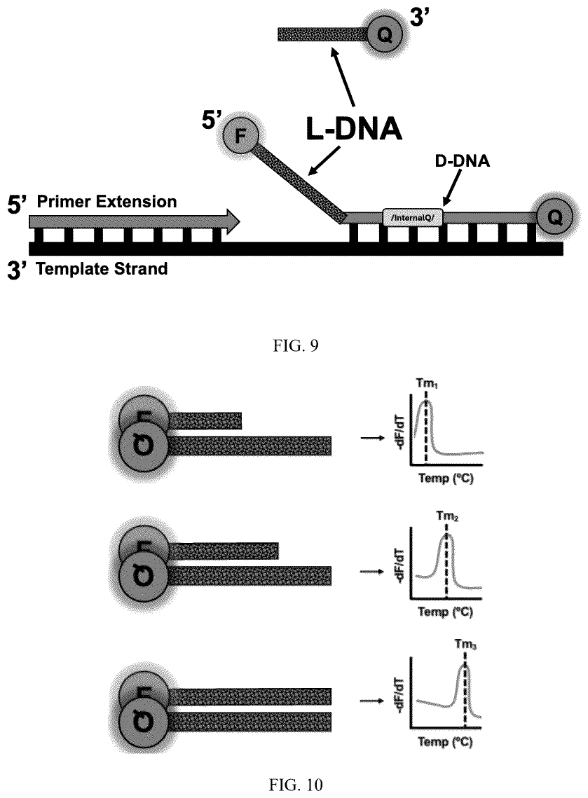

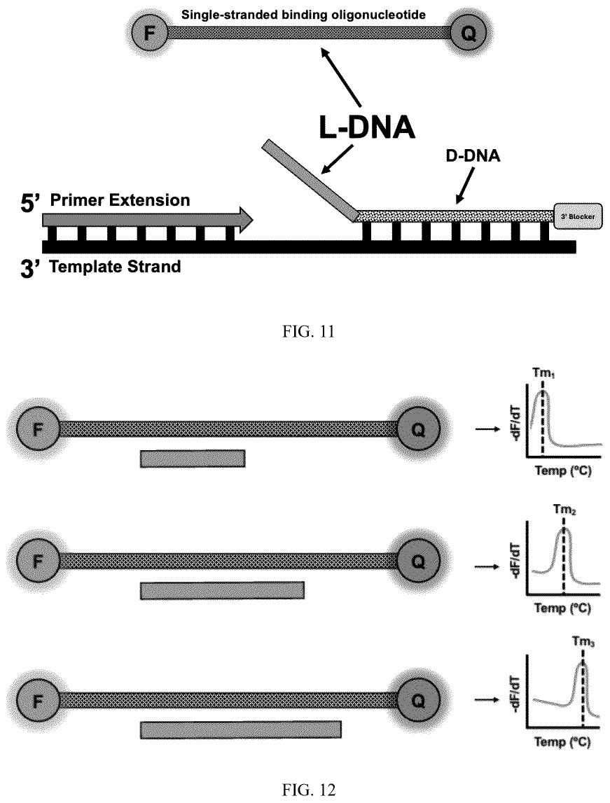

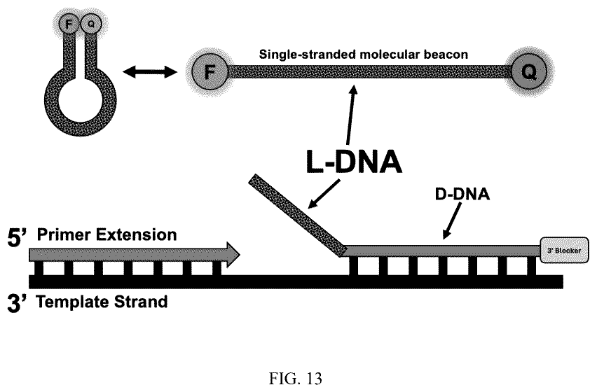

Polymerase chain reaction (PCR) is one of the most widely used diagnostic technologies due to its unparalleled sensitivity and specificity. Multiplex PCR enables simultaneous detection of multiple target sequences, broadening the diagnostic scope and conserving time and resources. Each additional PCR that can be added to a reaction increases the utility. However, assuming one could combine as may individual PCR reactions as desired into a single tube, state-of-the-art PCR instruments and detection reagents limit the maximum number of PCR reactions to one reaction for each of the instrument's fluorescence channels. To overcome these limitations, approaches for combining fluorescence color and sequence melting temperature (Tm) were previously explored. The strategy is limited by following the standard paradigm of one label (Tm-fluorescence) per one target analyte, and, therefore, restricts the number of uniquely discernable targets within a given design space to the number of unique Tm-fluorescence labels. In this work, it is aimed to counter this paradigm by increasing the number of uniquely identifiable targets within a given design space through label combinatorics. By selectively increasing the number of Tm-fluorescence labels for a given target, the multiple labels provide a combined, unique target-specific signature, referred to herein as a combinatoric code (FIG. 8A). This reimagined signature output strategy, achievable through relatively simple experimental modifications, significantly increases the number of target analytes within a given design space (FIG. 8B). This paradigm shift reduces design burden and reagent cost. Further, it improves the practicality of the platform genetic technology-requiring less Tm-fluorescence signals within a design space to reach high order multiplexability.

### Materials and Methods

The Tm-fluorescence signals are generated by modified PCR hydrolysis probes, called “mediator probes” consisting of a mediator sequence comprising 5′-flap sequence designed to be cleaved by the exonuclease activity of polymerase during amplification. Following cleavage, the 5′-flap hybridizes to a reporter probe (e.g., a molecular beacon) to produce a unique Tm-fluorescence output (i.e., a “signature element”). To generate multiple output labels, multiple mediator probes are designed for the same hybridization site on the target in competition. Here, how the competition-based labeling strategy impacts the analytical performance of the assay is studied. Competitive probes were designed to produce up to three unique labels in a single reaction for the detection of Influenza A. Analytical sensitivity and specificity were observed as the number of competitive probes increased from one to three across titered concentrations of spin-column (Qiagen, #52904) extracted Influenza A (BEI Resources, NR-15241), following a separate reverse transcription step. Reverse transcription was performed at 50° C. for 10 minutes with ProtoScript II (NEB, #M0368). PCR was performed on a QuantStudio 5 (Applied Biosystems) with GoTaq PCR Master Mix (Promega, #M7132) with the following cycling conditions: 95° C. for 10 min, 45 cycles of 95° C. for 15 s and 60° C. for 60 s. An extension step used to facilitate the interaction between the 5′-flap and the molecular beacon was run immediately following PCR with a 30-minute hold at 35° C. Melt analysis was performed from 45° C. to 95° C. with a ramp rate of 0.5° C./s. Positive detection was determined by clearly discernable signals at the expected label Tm-fluorescence following subtraction of the no template control signals.

### Results

By increasing the number of combined labels (k) for a single target, the number of unique targets increases from equal to the number of targets in a design space (n) to n choose k (nCk), which can be expressed as:

$$nCk = n ! / k ! ( n - k ) !$$

With a given design space of n=15 Tm-fluorescence possible signals, the standard paradigm of one signal per target has a maximum number of uniquely identifiable targets of 15. Increasing combined signals to k=2 and k=3 results in a maximum of 105 and 455 unique identifiers for a single target, respectively (FIG. 8B). Signals are increased by introducing mediator probes with varied 5′-flap sequences in combination. For a single probe (k=1), the combination PCR assay resulted in melt analysis-based detection for 6 of 6 extracted influenza A samples at 10¹ TCID₅₀/mL and 3 of 6 samples at 10⁰ TCID₅₀/mL. When an additional competitive probe was introduced (k=2), influenza A at 10¹ TCID₅₀/mL was detected 6 of 6 times and 10⁰ TCID₅₀/mL was detected in 1 of 6 replicates. For k=3, 6 of 6 replicates, 2 of 6 replicates, and 0 of 6 replicates were detected for 10² TCID₅₀/mL, 10¹ TCID₅₀/mL, and 10⁰ TCID₅₀/mL, respectively. Across all combinatoric studies, assay specificity was maintained with no false positives. Representative melt curves for one of the signals across number of mediator probes and titered influenza A concentration is demonstrated in FIG. 8C.

### Example 4. Fundamentals of Enantiomeric DNA Structures for Next Generation of Genetic Tools

What if nature's mirror, left-handed DNA (L-DNA), holds the key to genetic tools more precise than evolution ever needed? L-DNA is the non-superimposable mirror image, or enantiomer, of natural D-DNA. As such, L-DNA is thought to mimic the molecular characteristics of D-DNA while remaining unaffected by noise from natural enzymes and D-DNA background common in biological samples and genetic assays. Recent work with enantiomeric structures has demonstrated promising performance when coupled with genetic analysis tools, sparking the question: what other benefits might L-DNA bring to the next generation of analytical tools? This project focuses on communicating information from the natural D-DNA world to the optimal, unimpeded L-DNA space. Chimeric DNA structures, containing both D-DNA and L-DNA, provide this synergistic channel. L-DNA interactions and reporting from the L-DNA space, without D-DNA or enzymatic interference, enables the development of highly specific, ultra-sensitive, and versatile features in the next generation of genetic tools. Yet, the fundamentals underlying these enantiomeric technologies remain largely unexplored. The structural, behavioral, and functional properties of enantiomeric structures, with emphasis on hybrid L-/D-DNA chimeras are explored herein.

Novel PCR-based multi-bit methods, based on existing D-DNA technologies, that employs the enantiomeric specific features for enhanced capabilities is disclosed herein. The multi-bit PCR methods as disclosed herein have a robust capacity to detect hundreds of sequence-established signatures in a single reaction, expanding far beyond the capabilities of the gold-standard PCR technology. The multi-bit PCR methods as disclosed herein are enabled by a developed mechanism to synergistically link the natural D-DNA world and a dynamic L-DNA output that is unburdened by the large amount of D-DNA necessary for high order multiplexing.

The multi-bit PCR methods as disclosed herein are designed to reduce cost, improve flexibility/user friendliness, and speed up turnaround time when testing for multiple pathogens with similar symptomatic presentations. Yet, this project focuses on the development of a platform technology that can be used for genetic profiling extending far beyond the diagnostic clinic. By leveraging the largely unexplored world of L-DNA, the project has both the potential to achieve unprecedented access to advanced genetic technologies and remains unproven. The genetic platform tool designed with L-/D-DNA chimeric structures is a unique and innovative approach, employing cutting edge designs for nucleic acids. Within this design, the D-DNA section of the chimeric structures to specifically interact with the target(s) of interest and enable the L-DNA segment of the structure to be cleaved from the chimera, activating a unique, combinatoric response with L-DNA nucleic acid reporters.

### Methodologies

The overall objective of this project is to establish a chimeric L-/D-DNA-based platform that enables high-order multiplexed genetic detection in a single reaction, exceeding the capacity of gold-standard PCR technologies. It is aimed to (1) design and characterize L-/D-DNA chimeric structures capable of sequence-specific recognition of target nucleic acids (D-DNA) and orthogonal, interference-free reporting (L-DNA). It is also sought to (2) demonstrate the enzymatic compatibility and cleavage selectivity of the system, which would allow specific activation of L-DNA reporters in response to D-DNA hybridization. (3) It is further aimed to develop a multiplexed detection system that leverages the orthogonality and chemical stability of L-DNA to encode combinatoric signatures representative of up to hundreds of unique genetic targets within the same reaction. It is further sought to (4) validate analytical performance (e.g., sensitivity, specificity) in a clinically relevant panel involving pathogen detection from contrived samples in complex biological backgrounds.

Characterization studies consist of three, at times overlapping, classes: (1) structural, (2) behavioral, and (3) functional. For structural investigations, varied L-/D-DNA chimeric structures (e.g., 5′-LLLDDD-3′ and 5′-LLDDDLL-3′) are synthesized to determine synthesis and assembly limitations and measure impact on conformational structures, melt properties, and molecular stability. Behavioral studies investigate characteristics such as enantiomeric structure interaction with biostructures in solution (e.g., proteins) and biosafety of L-DNA based tools. The functional characteristic studies focus on the L-DNA and chimeric L-/D-DNA structures engagement with enzymatic processes (e.g., polymerase activity, CRISPR-Cas recognition, ligation), their interactions with DNA intercalators and end label structures, and the impacts of nuclease activity on the varied chimeras. Following characterization of the L-DNA and L-/D-DNA enantiomeric structures, it is aimed to develop a proof-of-concept genetic tool, based on existing D-DNA technologies, that employs the enantiomeric specific features for enhanced capabilities. Informed by studies, the multi-bit PCR methods as disclosed herein are modified using the L-/D-DNA enantiomeric structures, with a robust capacity to detect hundreds of sequence-established signatures in a single reaction, expanding far beyond the capabilities of the gold-standard PCR technology. This technology is enabled by a developed mechanism to synergistically link the natural D-DNA world and a dynamic L-DNA output that is unburdened by the large amount of D-DNA necessary for high order multiplexing.

The innovation is at the intersection of synthetic nucleic acid chemistry, structural biology, and molecular diagnostics. Individual enantiomers have been characterized; however, the structural, behavioral, and functional characteristics of hybrid chimeras have been largely unexplored. Understanding these characteristics is an essential challenge in the technology design. Another challenge exists in the application of enzymatic selectivity for L-DNA containing structures. Standard enzymes (e.g., polymerase) have evolved to interact with only D-DNA. Yet, methods disclosed in some non-limiting embodiments herein rely on the selective cleavage of the L-DNA to be triggered by enzymatic interaction with the D-DNA segment of the chimera. As such, the chimeras must be specifically designed to enable this enzyme interaction in a programmable fashion. Overcoming this challenge is necessary for reliable transduction between the L- and D-DNA domains. This synergistic link is at the crux of the potential of the L-/D-DNA chimeric method, promising a reliable platform genetic technology capable of high-order multiplexing in a single tube on a standard real-time PCR instrument.

In some embodiments, the platform technology disclosed herein takes an unconventional approach that uses D-DNA for specific, biological recognition and L-DNA for enhanced signal output. This approach decouples the sensing and signaling steps in genetic detection and, as such, enables multiplexed detection unimpeded by background DNA or enzymatic noise common in today's biological systems. Further, an approach to L-DNA signal coding employ combinatoric logic that near-exponentially expands the detection space-something impossible under the standard detection paradigm in current PCR-based systems. The competitive advantages unique to the methods disclosed herein are the ability to (1) to perform massively multiplexed detection with high specificity and low-cross talk, (2) to scale multiplexing with marginal increase in cost, and (3) operate in complex biological samples with high levels of D-DNA background.

This project addresses a critical unmet need in multiplex PCR/genetic technologies, where current methods are limited by reaction capacity, signal interference, and lack of adaptability. As a result, users must run multiple separate assays, increasing cost, turnaround time, and sample consumption—a major limitation for applications in both clinical and non-clinical settings. The L-/D-DNA chimeric methods described herein enable detection among hundreds of targets in a single reaction by decoupling recognition from signal output. The technology uses L-DNA reporters that are orthogonal to natural enzymes and background DNA, allowing for clean, multiplexed, interference-free signal output at a scale not possible with conventional real-time PCR. PCR users consistently report pain points such as limited target capacity per assay, high cost, and time spent per sample when running large panels, and difficulty modifying assay to adapt to new targets. In parallel, the platform is well-suited for agricultural genomics, veterinary diagnostics, and environmental monitoring, where users need affordable and scalable molecular tools to track genetic markers or pathogens.

TABLE 5
Molecular Beacon Sequences

| | | |
|---|---|---|
| Oligo Name | Sequence (5′ > 3′) | Notes |

Molecular Beacons

| | | |
|---|---|---|
| L- | 56-FAM- | Stem Tm = 50° C. |
| DJN001 | CGAGCAAAAAGAAGTGTGAGAGGTGTGATGAGCTCG- | |
| | BHQ1 (SEQ ID NO: 12) | |
| L- | SUN- | Stem Tm = |
| DJN002 | GCTGCAAAAAACTCAACGATGTGGAAGTCAGCAGC- | 53.9° C. |
| | BHQ2 (SEQ ID NO: 13) | |
| L- | C GAG CTC ATC ACA C (SEQ ID NO: 14) | Tm = 54.1° C. w/ |
| DJN003 | | L-DJN001 |
| L- | C GAG CTC ATC ACA CCT C (SEQ ID NO: 15) | Tm = 60.5° C. w/ |
| DJN004 | | L-DJN001 |
| L- | C GAG CTC ATC ACA CCT CTC ACA CTT | Tm = 68.8° C. w/ |
| DJN005 | (SEQ ID NO: 16) | L-DJN001 |
| L- | GC TGC TGA CTT CCA (SEQ ID NO: 17) | Tm = 56.4° C. w/ |
| DJN006 | | L-DJN002 |
| L- | G CTG CTG ACT TCC ACA TCG (SEQ ID NO: 18) | Tm =64.3° C. w/ |
| DJN007 | | L-DJN002 |
| L- | G CTG CTG ACT TCC ACA TCG TTG AGT | Tm = 69.6° C. w/ |
| DJN008 | (SEQ ID NO: 19) | L-DJN002 |

All materials in the above table are L-DNA (also underlined)

TABLE 6
Mediator Probe and Reporter Sequences

| | | |
|---|---|---|
| Oligo Name | Sequence (5′ > 3′) | Notes |

Quencher Beacons

| | | |
|---|---|---|
| L- | AAG TGT GAG AGG TGT GAT GAG CTC G/BHQ1/ | Tm = |
| DJN001-A | (SEQ ID NO: 20) | 68.8° C. |
| L- | G CTG CTG ACT TCC ACA TCG TTG AGT/BHQ1/ | Tm = |
| DJN002-B | (SEQ ID NO: 21) | 69.6° C. |

Mediator Primer Beacon Pairs

| | | |
|---|---|---|
| L- | /56-56-FAM/C GAG CTC ATC ACA C (SEQ ID NO: 22) | Tm = |
| DJN003-A1 | | 54.1° C. |
| | | w/L- |
| | | DJN001 |
| L- | /56-56-FAM/C GAG CTC ATC ACA CCT C (SEQ ID NO: 23) | Tm = |
| DJN004-A2 | | 60.5° C. |
| | | w/L- |
| | | DJN001 |
| L- | /56-FAM/C GAG CTC ATC ACA CCT CTC ACA CTT (SEQ ID NO: 24) | Tm = |
| DJN005-A3 | | 68.8° C. |
| | | w/L- |
| | | DJN001 |
| L- | /SUN/ACT CAA CGA TGT GG (SEQ ID NO: 25) | Tm = |
| DJN006-B1 | | 53.2° C. |
| | | w/L- |
| | | DJN002 |
| L- | /SUN/ACT CAA CGA TGT GGA AGT C (SEQ ID NO: 26) | Tm = |
| DJN007-B2 | | 61.1° C. |
| | | w/L- |
| | | DJN002 |
| L- | /SUN/ACT CAA CGA TGT GGA AGT CAG CAG C (SEQ ID NO: 27) | Tm = |
| DJN008-B3 | | 69.6° C. |
| | | w/L- |
| | | DJN002 |

Mediator Probes

| | | |
|---|---|---|
| L- | /56-FAM/C GAG CTC ATC ACA CCT C | |
| DJN009 | CTTG/ZEN/TCACCTCTGACTAAGGGAATTTTAGGAT/3IABKFQ/ | |
| \|Flu A\| | (SEQ ID NO: 28) | |
| A-2 | | |
| L- | /56-FAM/C GAG CTC ATC ACA CCT CTC ACA CTT | |
| DJN010 | CTTG/ZEN/TCACCTCTGACTAAGGGAATTTTAGGAT/3IABKFQ/ | |
| \|Flu A\| | (SEQ ID NO: 29) | |
| A-3 | | |
| L- | /56-FAM/C GAG CTC ATC ACA CCT C | |
| DJN011 | GCTA/ZEN/TTGTGCACTAARGATATTTGATGAGAAGTAGT/3IAB | |
| \|RSV A\| | kFQ/ | |
| A-2 | (SEQ ID NO: 30) | |
| L- | /SUN/ACT CAA CGA TGT GGA AGT C | |
| DJN012 | | |
| \|RSV A\| | GCTA/ZEN/TTGTGCACTAARGATATTTGATGAGAAGTAGT/3IAB | |
| B-2 | kFQ/ | |
| | (SEQ ID NO: 31) | |
| L- | /56-FAM/C GAG CTC ATC ACA CCT C | |
| DJN013 | ACCC/ZEN/CGCATTACGTTTGGTGGACC/3IABKFQ/ | |
| \|SC2\| | (SEQ ID NO: 32) | |
| A-2 | | |
| L- | /SUN/ACT CAA CGA TGT GG | |
| DJN014 | ACCC/ZEN/CGCATTACGTTTGGTGGACC/3IABKFQ/ | |
| \|SC2\| | (SEQ ID NO: 33) | |
| B-1 | | |
| L- | /56-FAM/C GAG CTC ATC ACA CCT CTC ACA CTT | |
| DJN015 | CCTC/ZEN/CGGCCCCTGAATGYGGCTAAYC/3IABKFQ/ | |
| \|EV/RV\| | (SEQ ID NO: 34) | |
| A-3 | | |
| L- | /SUN/ACT CAA CGA TGT GGA AGT C | |
| DJN016 | CCTC/ZEN/CGGCCCCTGAATGYGGCTAAYC/3IABKFQ/ | |
| \|EV/RV\| | (SEQ ID NO: 35) | |
| B-2 | | |
| L- | /SUN/ACT CAA CGA TGT GG | |
| DJN017 | TATT/ZEN/CCTTACTAAAGATGTCTGATKGGAAGTGGTGG/3IA | |
| \|RSV B\| | BkFQ/ | |
| B-1 | (SEQ ID NO: 36) | |
| L- | /SUN/ACT CAA CGA TGT GGA AGT C | |
| DJN018 | TATT/ZEN/CCTTACTAAAGATGTCTGATKGGAAGTGGTGG/3IA | |
| \|RSV B\| | BkFQ/ | |
| B-2 | (SEQ ID NO: 37) | |
| L- | /56-FAM/C GAG CTC ATC ACA C | |
| DJN019 | CGAG/ZEN/CAGCTGAAACTGCGGTGGGAG/3IABKFQ/ | |
| \|Flu B\| | (SEQ ID NO: 38) | |
| A-1 | | |
| L- | /SUN/ACT CAA CGA TGT GGA AGT C | |
| DJN020 | CGAG/ZEN/CAGCTGAAACTGCGGTGGGAG/3IABKFQ/ | |
| \|Flu B\| | (SEQ ID NO: 39) | |
| B-2 | | |
| L- | /56-FAM/C GAG CTC ATC ACA CCT CTC ACA CTT | |
| DJN021 | AACY/ZEN/GCAGTGACACCYTCATCATTGCAGCAAG/3IABKFQ | |
| \|HMPV\| | (SEQ ID NO: 40) | |
| A-3 | | |
| L- | /SUN/ACT CAA CGA TGT GG | |
| DJN022 | AACY/ZEN/GCAGTGACACCYTCATCATTGCAGCAAG/3IABKFQ/ | |
| \|HMPV\| | (SEQ ID NO: 41) | |
| B-1 | | |
| L- | /56-FAM/C GAG CTC ATC ACA C | Based |
| DJN023 | CGCT/ZEN/AACGGCAACACGTAATCAGGTCA/3IABKFQ/ | on |
| \|M. | (SEQ ID NO: 42) | DJN244 |
| Pneumo\| | | |
| A-1 | | |
| L- | /56-FAM/C GAG CTC ATC ACA CCT C | Based |
| DJN024 | CGCT/ZEN/AACGGCAACACGTAATCAGGTCA/3IABKFQ/ | on |
| \|M. | (SEQ ID NO: 43) | DJN244 |
| Pneumo\| | | |
| A-2 | | |
| L- | /56-FAM/C GAG CTC ATC ACA C | Based |
| DJN025 | GAAT/ZEN/GGCAAGTAGGAGCCTCTCTATCTTA/3IABKFQ/ | on |
| \|C. | (SEQ ID NO: 44) | DJN247 |
| Pneumo\| | | |
| \|A-1 | | |
| L- | /SUN/ACT CAA CGA TGT GG C | Based |
| DJN026 | GAAT/ZEN/GGCAAGTAGGAGCCTCTCTATCTTA/3IABKFQ/ | on |
| \|C. | (SEQ ID NO: 45) | DJN247 |
| Pneumo\| | | |
| \|B-1 | | |
| L- | /56-FAM/C GAG CTC ATC ACA C | Based |
| DJN027 | CAAT/ZEN/CCAACACGGCATGAACGCTC/3IABKFQ/ | on |
| \|B. Pert\| | (SEQ ID NO: 46) | DJN250 |
| A-1 | | |
| L- | /56-FAM/C GAG CTC ATC ACA CCT CTC ACA CTT | Based |
| DJN026 | CAAT/ZEN/CCAACACGGCATGAACGCTC/3IABKFQ/ | on |
| \|B. Pert\| | (SEQ ID NO: 47) | DJN250 |
| A-3 | | |
| L- | /SUN/ACT CAA CGA TGT GGA AGT CAG CAG C | |
| DJN028 | TCTG/ZEN/ACCTGAAGGCTCTGCGCG/3IABKFQ/ | |
| \|RNaseP\| | (SEQ ID NO: 48) | |
| B-3 | | |

Chimeric Structures-Underlined = L-DNA

### Example 5. Highly Scalable, Combinatoric PCR Strategy Enables Augmented Panel-Based Multiplexing for Target Detection

### Combinatorics Unlock Exponential Number of Targets Detectable in a Single Reaction

Multiplex polymerase chain reaction (PCR) enables simultaneous detection of multiple pathogen target sequences, broadening the diagnostic scope and conserving time and resources. Each additional PCR that can be added to a reaction increases the utility of this gold standard approach. It is aimed to exponentially increase the number of uniquely identifiable targets within a given detection space through label combinatorics.

As exemplified in FIGS. 8A-8B, design space (n) demonstrates an example of 15 melt temperature (Tm) and fluorescence combined labels. The existing approaches follow the standard paradigm of one output label for the detection of one target analyte (k=1). As disclosed herein, multi-bit PCR utilizes multiple Tm-fluorescence labels in combination for detection of a single target, increasing the maximum detectable targets to n choose k, or 15 choose 2 or 3 in the pictured example. The combinatoric approach exponentially increases the number of uniquely discernable targets within a given design space of the number, n, Tm and fluorescence signature elements.

By using more than one melt temperature (Tm)-fluorescence label for any given target, the multiple labels provide a combined, unique target-specific signature.

### Polymerase Exonuclease Activity Enables Fluorescence-Melt Temperature Signatures

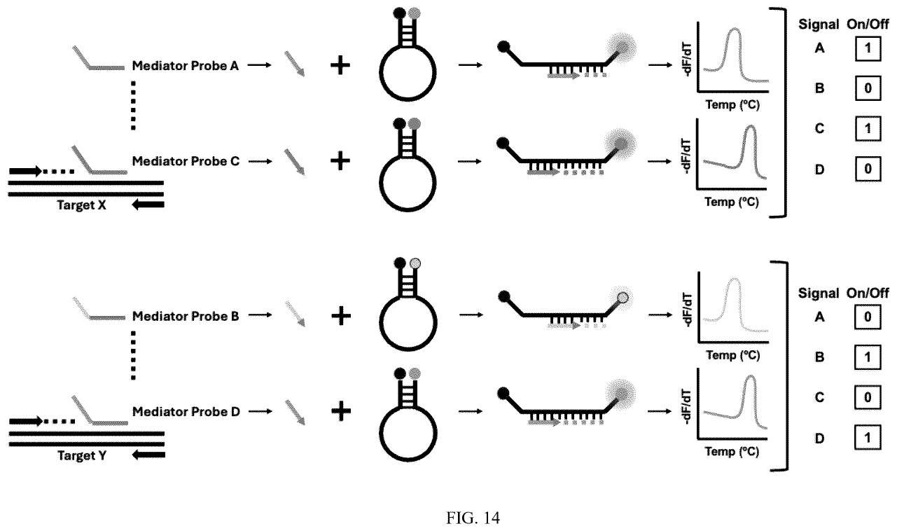

The combinatoric PCR strategy is achieved by 5′-flap cleavage during the polymerase extension step of PCR (FIG. 14). Mediator probes consisting of a target-specific region and a specific 5′ sequence called a mediator primer (colored) are used as a hydrolysis probe during PCR (1). Following cleavage, the mediator primers hybridize to and extend along molecular beacons (2). Extended products generate melt temperatures (Tm) with the beacon specific fluorescence. Tm-fluorescence signals are used in combination as target-unique signatures (3).

### Probe Design Approaches for Single Target Combinatoric Labeling

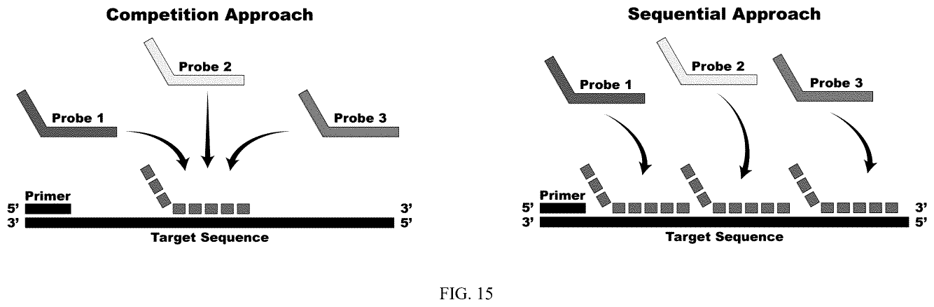

Multiple mediator probes corresponding to different signal outputs can be used in combination, by either competition or sequential approaches, to exponentially increase the number of discernable targets within a single tube (FIG. 15). Mediator probes are designed to hybridize with the same target specific sequence while each containing unique 5′-flap sequences for generating Tm-fluorescence signals.

### Single Target Combinatorics are Achieved by Probe Design Modularity

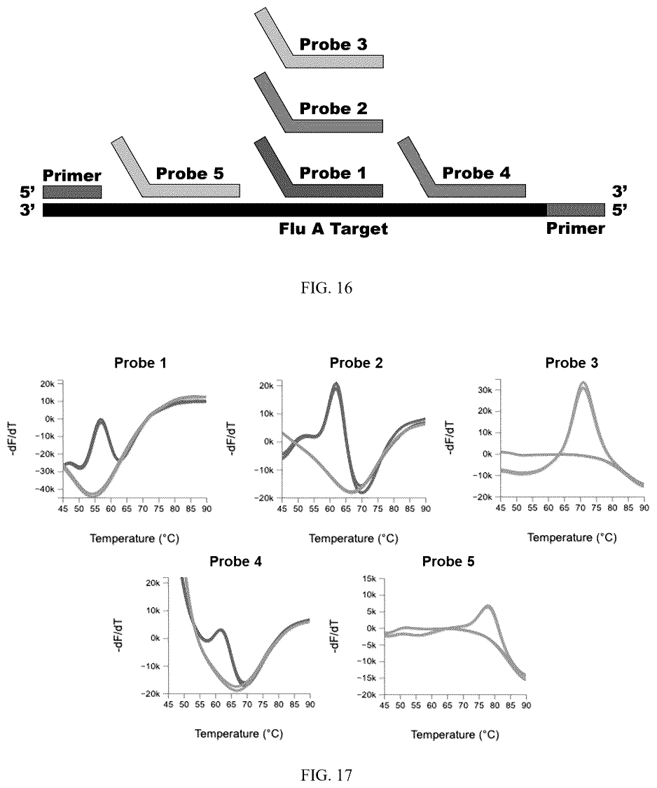

Mediator probes are designed by appending beacon-specific mediator primers to target-specific hydrolysis probes (FIG. 16). For example, five mediator probes were designed for targeting Influenza A, each with unique mediator primers, to investigate the competition versus sequential combinatoric approaches.

The five Influenza A specific mediator probes detects the virus specifically and with unique Tm-fluorescence outputs (FIG. 17). For these studies, the mediator probes were used individually for detection of Influenza A and compared to no target controls.

Competition and Sequential Combinatoric Approaches detect Influenza A Titrations

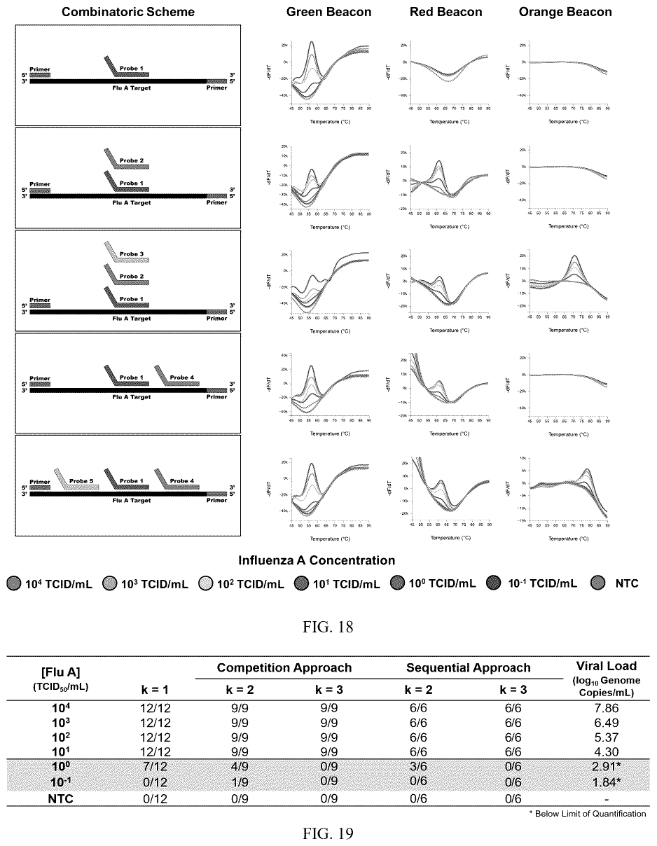

The combinatoric approaches each successfully detect Influenza A positive samples across titrated target concentrations (FIG. 18). Combinatoric probe schemes varying the number of probes used (k=1, 2, or 3) and the competition (stacked) or sequential (side-by-side) approaches were tested. Raw data is shown for each of the three molecular beacons employed for the combinatoric signal output, with the curves in the raw data correspond to the respective concentrations (FIG. 18).

The Combinatoric Approaches Perform with Similar Analytical Sensitivity

A summary of the analytical performance of the probe design approaches with different number of probes (k) is shown in FIG. 19. Influenza A virus (BEI NR-15241, (H1N1) pdm09, San Diego) was extracted and titrated. Following a separate reverse transcriptase step, the combinatoric approaches with k=2 and k=3 probes each were investigated for their analytical sensitivity. The shaded area is below the assay limit of detection. Viral load was determined by Hologic Panther RT-PCR with Influenza A quantitative standards.

Combinatoric Approach enables Future Highly Multiplex Assay with Minimal Reporters

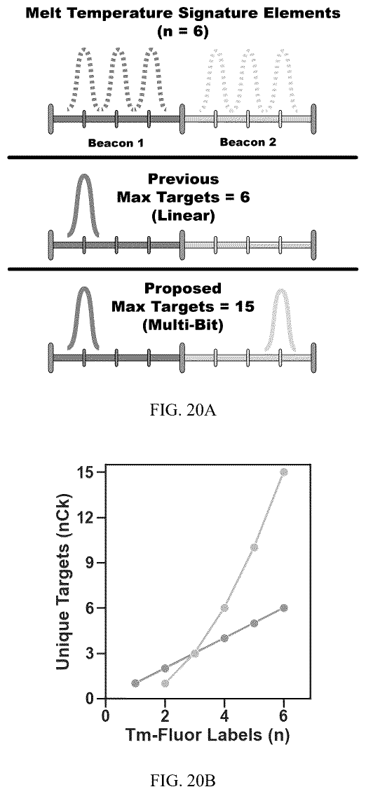

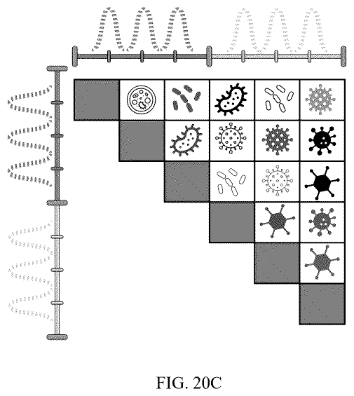

The combinatoric approach has practical utility even with reduced label design space (FIGS. 20A-20C). A simplified approach with two beacons, each with three robustly-spaced melt temperatures, for a proof-of-concept 6 choose 2 assay (FIG. 20A). The number of unique targets detectable measurably increases within the simplified assay as compared to the standard approach (FIG. 20B). An exemplary design grid demonstrates the 15-plex assay achievable through the 6 choose 2 combinatorics (FIG. 20C).

This assay design is practical for panel-based assays, such as an in-development respiratory panel.

### CONCLUSIONS

(i) Combinatoric codes comprising multiple designable Tm-fluorescence signals exponentially increases the number of unique targets detectable in a single PCR assay;
(ii) The combinatoric PCR strategies disclosed herein are capable of being performed in a single tube on standard real-time instrumentation;
(iii) The modularity of mediator probe designs enable target and assay flexibility;
(iv) The combinatoric PCR assay methods described herein performs similarly across the various approaches and different probe amounts up to the detection limits; and
(v) Combinatoric PCR provides a practical pathway for highly-multiplex panel-based assays, even with reduced reagents.

What is claimed is:

1. A method for detecting one or more target nucleic acid sequences in a sample by polymerase chain reaction (PCR) assay, comprising:
(i) providing two or more mediator probes for each target nucleic acid sequence, wherein each of the two or more mediator probes comprise (a) a non-complementary mediator sequence, and (b) a target specific sequence which is complementary to a target binding site;
(ii) contacting the sample, under conditions allowing for nucleic acid hybridization, with the mediator probes of step (i);
(iii) contacting products of step (ii) with an enzyme having 5′ exonuclease activity to yield one or more 5′ flap sequences derived from the non-complementary mediator sequence;
(iv) providing one or more detection probes and, under conditions allowing for nucleic acid hybridization, contacting the one or more 5′ flap sequences of step (iii) with the one or more detection probes;
wherein each of the one or more detection probes comprises (a) a capture sequence complementary to the 5′ flap sequence, or a fragment thereof, and (b) a templating sequence;
wherein each of the one or more detection probes are independently labeled with a reporter group capable of generating one or more signals in one or more fluorophore color channels;
(v) contacting products of step (iv) with a nucleic acid polymerase to generate unique length double-stranded products;
wherein each of the target nucleic acid sequences has a unique combinatoric code, wherein the unique combinatoric code comprises two or more signature elements each corresponding to a melting temperature (Tm) in a fluorophore color channel, and wherein the two or more signature elements are analyzable by the melting curve of the unique length double-stranded products of step (v) and the one or more signals of the reporter group of the detection probe;
(vi) subjecting the unique length double-stranded products of step (v) to a melt and fluorescence analysis to determine a fluorescence-melting temperature (F-Tm) signature element in each of the one or more fluorophore color channels for each of the one or more target nucleic acid sequences; and
(vii) determining, according to the results of the melt and fluorescence analysis in step (vi), whether each of the one or more target nucleic acid sequences are present in the sample.

2. The method of claim 1, further comprising the step of determining the amount of each of the one or more target nucleic acid sequences corresponding to each melting peak.

3. The method of claim 1, wherein the melt and fluorescence analysis of step (vi) comprises gradual heating and/or cooling of the unique length double-stranded products of step (v), wherein the one or more signals from the reporter group in each of the one or more detection probes is real-time monitored to provide the melting curve of each of the unique length double-stranded products of step (v), wherein identification of the F-Tm signature element for a unique length double-stranded product is determined according to a certain melting peak in the one or more fluorophore color channels, and wherein identification of the F-Tm signature element for an individual target nucleic acid sequence determines the presence of the individual target nucleic acid sequence in the sample.

4. The method of claim 1, wherein the one or more detection probes have a structure selected from a linear structure, a hairpin structure, a branching structure, a dendritic structure, a combination of hairpin and linear structure in a single strand, two strands in a molecular beacon double stranded DNA (dsDNA) structure, or a combination thereof.

5. The method of claim 1, wherein the one or more target nucleic acid sequences comprise DNA, RNA, or a mixture thereof.

6. The method of claim 1, wherein the one or more detection probes comprise reporter groups capable of generating a signal in 1, 2, 3, 4, or more than 4 fluorophore color channels.

7. The method of claim 1, wherein the one or more detection probes comprise reporter groups capable of generating 2, 3, 4, or more than 4 signals in each fluorophore color channel.

8. The method of claim 1, wherein several mediator probes are provided for a single target binding site of a target nucleic acid sequence.

9. The method of claim 1, wherein several mediator probes are provided for several different target binding sites within the same target nucleic acid sequence.

10. The method of claim 1, wherein the enzyme having 5′ exonuclease activity is a DNA polymerase derived from a bacterium selected from Thermus aquaticus (Taq), Thermus thermophiles (Tth), Thermus filiformis, Thermis flavus, Thermococcus literalis, Thermus antranildanii, Thermus caldophllus, Thermus chliarophilus, Thermus flavus, Thermus igniterrae, Thermus lacteus, Thermus oshimai, Thermus ruber, Thermus rubens, Thermus scotoductus, Thermus silvanus, Thermus thermophilus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosipho africanus, Pyrococcus woesei, Pyrococcus horikoshii, Pyrococcus abyssi, Pyrodictium occultum, Aquifexpyrophilus or Aquifex aeolieus.

11. The method of claim 1, wherein the reporter group is selected from 5′ 6-FAM, acridine orange, ALEX-350, CAL Fluor® Gold 540, CAL Fluor Orange 560, CAL Fluor Red 590, CAL Fluor Red 610, CAL Fluor Red 635, CY3, CY5, CY5.5, ethidium bromide, EvaGreen, EvaRuby, FAM, HEX, JOE, MAX, PicoGreen, Quasar 670, Quasar 705, ROX, SUN, SYBR gold, SYBR green, SYBR Safe, SYTO-9, SYTO-64, SYTO-82, TAMRA, TET, TEXAS RED, thiazole orange, TOTO-1, VIC, Yakima Yellow, or YOYO-1.

12. The method of claim 1, wherein each of the target-complementary specific sequences are independently conjugated at a 5′ or 3′ terminus with a Homo-Tag Assisted Non-Dimer System (HANDS) tag primer.

13. The method of claim 1, wherein the method is performed in a single tube.

14. The method of claim 1, wherein each of the two or more mediator probes independently comprise a chimera of D-DNA and L-DNA or D-RNA and L-RNA.

15. The method of claim 1, wherein the non-complementary mediator sequence, the 5′ flap sequence, and/or the one or more detection probes comprise L-DNA residues.

16. The method of claim 1, wherein the sample is selected from a nasal swab, blood, plasma, serum, urine, sputum, spinal fluid, cerebrospinal fluid, pleural fluid, nipple aspirate, lymph fluid, respiratory tract fluid, intestinal tract fluid, genitourinary tract fluid, tear fluid, saliva, breast milk, lymphatic system fluid, semen, ascitic fluid, tumor cyst fluid, amniotic fluid, tissue, biopsy, feces, or a combination thereof.

17. A method for determining presence and/or amount of one or more target nucleic acid sequences derived from a virus or a bacterium in a sample, comprising:
(i) designing one or more target specific sequences which are complementary to a target binding site in the one or more target nucleic acid sequences; and
(ii) conducting the method of claim 1 using two or more mediator probes comprising the one or more target specific sequences of step (i).

18. The method of claim 17, wherein the virus is selected from Adenovirus, Bocavirus, SARS-CoV-2, Cytomegalovirus, Dengue virus, Influenza virus A, Influenza virus B, Hepatitis virus, Herpes Simplex Virus (HSV), human immunodeficiency virus (HIV), human metapneumovirus (hMPV), Human Papillomavirus (HPV), Norovirus, Orthopoxvirus, Parainfluenza virus, Respiratory Syncytial Virus (RSV), Rhinovirus, or Varicella-zoster viruses.

19. The method of claim 17, wherein the bacterium is selected from Streptococcus pneumoniae, Streptococcus pyogenes, Mycoplasma pneumoniae, Mycoplasma genitalium, Haemophilus influenzae, Stephylococcus aureus, MRSA, Bordetella pertussis, Chlamydia pneumonia, Chlamydia trachomatis, Klebsiella pneumoniae, Bacillus Anthracis, Candida auris, Candida tropicalis, Candida albicans, Candida parapsilosiis, Candida glabrata, Candida kruseii, Trichomonas vaginalis, Neisseria gonorrhoeae, Clostridium difficile, Coxiella burnetii, Entercoccus, Escherichia coli, Pseudomonas aeruginosa, Francisella tularensis, Mycobacterium tuberculosis, Streptococci spp., Salmonella spp., Cryptococcus neoformans, Legionella pneumophila, or Yersinia pestis.

20. A method for determining presence of one or more single-nucleotide polymorphisms (SNPs) in one or more target nucleic acid sequences in a sample, comprising:
(i) designing one or more target specific sequences which are complementary to a target binding site comprising a potential SNP in the one or more target nucleic acid sequences; and
(ii) conducting the method of claim 1 using two or more mediator probes comprising the one or more target specific sequences of step (i).
